(19) United States
(12) Patent Application Publication
(10) Pub. No.: US 2005/0196797 A1
(43) Pub. Date: Sep. 8, 2005

(54) SMG-1 binding protein and screening method for substances that can regulate the activity thereof

(75) Inventors: Shigeo Ohno, Tokyo (JP); Tetsuo Ohnishi, Kanagawa (JP); Akio Yamashita, Kanagawa (JP)

Correspondence Address: HESLIN ROTHENBERG FARLEY & MESITI PC, 5 COLUMBIA CIRCLE, ALBANY, NY, 12203, US

(73) Assignees: Japan Science and Technology Agency, Saitama (JP); City of Yokohama, Kanagawa (JP)

(21) Appl. No.: 11/072,699

(22) Filed: Mar. 4, 2005

Related U.S. Application Data
(63) Continuation-in-part of application No. PCT/JP03/11353, filed on Sep. 5, 2003.

(30) Foreign Application Priority Data
Sep. 5, 2002 (JP) 2002-260243

(51) Int. Cl. C12Q 1/68; A01K 67/00; C07H 21/04; C07K 14/705; C07K 16/28

(52) U.S. Cl. 435/6; 435/69.1; 435/320.1; 435/325; 530/350; 530/388.26; 536/23.5; 800/8

(57) ABSTRACT

A novel polypeptide binding to SMG-1, a polynucleotide encoding the same, and a system for screening a substance which artificially modifies an mRNA surveillance mechanism, are disclosed. The polypeptide has an activity of binding to SMG-1, an activity of being phosphorylated by SMG-1, and an activity of modifying SMG-1 activities through these activities.

### CROSS REFERENCE TO RELATED APPLICATION

This is a continuation-in-part application of International Application No. PCT/JP03/011353 filed on Sep. 5, 2003.

### TECHNICAL FIELD

The present invention relates to an SMG-1 binding protein and a screening method for substances that can regulate or modify the activity thereof.

### BACKGROUND ART

Approximately one-quarter of abnormal phenotypes due to gene mutations are thought to be caused by a nonsense codon generated in a coding region, due to one or more mutations such as substitution, recombination, and/or deletion in the nucleotide sequence of the corresponding gene. The gene with such a nonsense codon encodes an abnormal protein lacking the C-terminal region, but no mRNA encoding the abnormal protein is usually detected. This is because cells have a mechanism in which mRNAs having one or more nonsense codons at abnormal positions are recognized and removed. The mechanism is called nonsense-mediated mRNA decay (or mRNA surveillance), and it is suggested that the mechanism is closely involved in human genetic diseases, and plays a physiological role in the removal of abnormal mRNAs generated when immunocompetent cells are matured. However, the molecular mechanism has not been clarified in vertebrates.

The present inventors and coworkers revealed and reported that phosphorylation of human UPF1 by a protein kinase molecule, human SMG-1 (hSMG-1), is essential to a process of mRNA surveillance [Yamashita, Akio, et al, “GENES & DEVELOPMENT”, (USA), 2001, 15, p. 2215-2228 (non patent reference 1)]. However, a surveillance complex containing human SMG-1 has not been clarified.

In this connection, a nucleotide sequence of a human cDNA encoding a novel polypeptide found in the present invention was reported in Kawabata, A., Hikiji, T., Kobatake, N., Inagaki, H., Ikema, Y., Okamoto, S., Okitani, R., Ota, T., Suzuki, Y., Obayashi, M., Nishi, T., Shibahara, T., Tanaka, T., Nakamura, Y., Isogai, T., and Sugano, S., “NEDO human cDNA sequencing project.” Homo sapiens cDNA . . . [gi:10439815]. Datasheet. [online]. Entrez Nucleotides database, National Center for Biotechnology Information. Entrez accession No: AK026858. [retrieved on 2002-09-02]. Retrieved from the Internet: <URL: http://www.ncbi.nlm.nih.gov:80/entrez/query.fcgi?cmd=Retrieve&db=nucleotide&list_uids=10439815&dop t=GenBank>(non patent reference 2). However, the reference does not disclose that the polypeptide was obtained by expressing the cDNA, and does not disclose the functions of the polypeptide.

Further, protein p140 having a molecular weight similar to that of the novel polypeptide found in the present invention was disclosed in Onishi, et al., “Analysis of structure and functions of novel PI3K-related huge protein kinase”, “PROGRAM AND ABSTRACTS IN THE 22ND ANNUAL MEETING OF THE MOLECULAR BIOLOGY SOClETY OF JAPAN”, 1999, p. 235 (non patent reference 3). However, the reference merely discloses human phosphatidyl inositol kinase (PIK)-related protein kinase (PIKK), which plays an important role in cell cycle progression or a regulation of checkpoints, but neither discloses nor suggests the mRNA surveillance mechanism.
(non patent reference 1) Yamashita, Akio, et al, “GENES & DEVELOPMENT”, (USA), 2001, 15, p. 2215-2228
(non patent reference 2) Kawabata, A., Hikiji, T., Kobatake, N., Inagaki, H., Ikema, Y., Okamoto, S., Okitani, R., Ota, T., Suzuki, Y., Obayashi, M., Nishi, T., Shibahara, T., Tanaka, T., Nakamura, Y., Isogai, T., and Sugano, S., “NEDO human cDNA sequencing project.” Homo sapiens cDNA . . . [gi: 10439815]. Datasheet. [online]. Entrez Nucleotides database, National Center for Biotechnology Information. Entrez accession No: AK026858. [retrieved on 2002-09-02]. Retrieved from the Internet: <URL: http://www.ncbi.nlm.nih. gov:80/entrez/query.fcgi?cmd=Retrieve&db=nucleotide&list_uid s=10439815&dopt=GenBank>
(non patent reference 3) Onishi, et al., “Analysis of structure and functions of novel PI3K-related huge protein kinase”, “PROGRAM AND ABSTRACTS IN THE 22ND ANNUAL MEETING OF THE MOLECULAR BIOLOGY SOClETY OF JAPAN”, 1999, p. 235.

### DISCLOSURE OF INVENTION

The present inventors engaged in intensive search with the object of elucidating an mRNA surveillance mechanism and developing a method capable of artificially modifying the mRNA surveillance mechanism, and as a result, identified a novel protein firmly binding to hSMG-1, and determined the full-length sequence thereof. The molecule firmly binds to hSMG-1 in cells, and thus, it is predicted that the molecule is closely involved in the modification of a kinase activity, intracellular localization, and the modification of an activity of a surveillance complex. Therefore, the present inventors found that the novel protein is a candidate for the molecule target to artificially modify the mRNA surveillance mechanism. The present invention is based on these findings.

Therefore, an object of the present invention is to provide a novel polypeptide which binds to SMG-1, a polynucleotide encoding the polypeptide, and a system for screening substances which artificially modify the mRNA surveillance mechanism.

The present invention relates to (1) a polypeptide consisting of the amino acid sequence of SEQ ID NO: 2, (2) a polypeptide exhibiting an SMGBP1 activity and comprising the amino acid sequence of SEQ ID NO: 2, (3) a polypeptide exhibiting an SMGBP1 activity and comprising an amino acid sequence in which one or plural amino acids are deleted, substituted, and/or inserted at one or plural positions in the amino acid sequence of SEQ ID NO: 2, or (4) a polypeptide exhibiting an SMGBP1 activity and comprising an amino acid sequence having a 90% or more homology with that of SEQ ID NO: 2.

Further, the present invention relates to a polynucleotide encoding the polypeptide.

Further, the present invention relates to an expression vector comprising the polynucleotide.

Further, the present invention relates to a transformant comprising the polynucleotide.

Further, the present invention relates to an antibody or a fragment thereof, which binds to the above polypeptide.

Further, the present invention relates to a knockout non-human animal or a cell thereof, wherein an expression of a gene encoding the above polypeptide is partially or completely suppressed.

Further, the present invention relates to a method for screening a substance which modifies an SGMBP1 activity of the above polypeptide, comprising the steps of:
(1) bringing the polypeptide and SMG-1 into contact with a substance to be tested, under conditions such that the polypeptide exhibits the SMGBP1 activity in the absence of the substance to be tested, and
(2) analyzing whether or not the polypeptide exhibits the SMGBP1 activity.

Further, the present invention relates to a method for screening a substance which modifies an SGMBP1 activity of the above polypeptide, comprising the steps of:
(1) bringing a cell capable of producing the above polypeptide and SMG-1 into contact with a substance to be tested, under conditions such that the cell produces the polypeptide in the absence of the substance to be tested, and
(2) analyzing whether or not the polypeptide exhibits the SMGBP1 activity.

Further, the present invention relates to an agent for suppressing nonsense-mediated mRNA decay, comprising, as an active ingredient, a substance which modifies an SMGBP1 activity of the above polypeptide (for example, a substance obtained by the above screening method and modifying an SMGBP1 activity of the above polypeptide).

Further, the present invention relates to an agent for treating and/or preventing a disease caused by a premature translation termination codon generated by a nonsense mutation, comprising, as an active ingredient, a substance which modifies an SMGBP1 activity of the above polypeptide (for example, a substance obtained by the above screening method and modifying an SMGBP1 activity of the above polypeptide).

Further, the present invention relates to an agent for promoting nonsense-mediated mRNA decay, comprising, as an active ingredient, a substance which modifies an SMGBP1 activity of the above polypeptide (for example, a substance obtained by the above screening method and modifying an SMGBP1 activity of the above polypeptide).

Further, the present invention relates to a method for suppressing nonsense-mediated mRNA decay, comprising administering to a subject in need thereof a substance which modifies an SMGBP1 activity of the above polypeptide, in an amount effective therefor.

Further, the present invention relates to a method for treating and/or preventing a disease caused by a premature translation termination codon generated by a nonsense mutation, comprising administering to a subject in need thereof a substance which modifies an SMGBP1 activity of the above polypeptide, in an amount effective therefor.

Further, the present invention relates to a method for promoting nonsense-mediated mRNA decay, comprising administering to a subject in need thereof a substance which modifies an SMGBP1 activity of the above polypeptide, in an amount effective therefor.

Further, the present invention relates to use of a substance which modifies an SMGBP1 activity of the above polypeptide, in the manufacture of an agent for suppressing nonsense-mediated mRNA decay.

Further, the present invention relates to use of a substance which modifies an SMGBP1 activity of the above polypeptide, in the manufacture of an agent for treating and/or preventing a disease caused by a premature translation termination codon generated by a nonsense mutation.

Further, the present invention relates to use of a substance which modifies an SMGBP1 activity of the above polypeptide, in the manufacture of an agent for promoting nonsense-mediated mRNA decay.

Further, the present invention relates to an agent for suppressing nonsense-mediated mRNA decay, comprising, as an active ingredient, a substance which suppresses an SMGBP1 activity of the above polypeptide.

Further, the present invention relates to an agent for treating and/or preventing a disease caused by a premature translation termination codon generated by a nonsense mutation, comprising, as an active ingredient, a substance which suppresses an SMGBP1 activity of the above polypeptide.

According to a preferred embodiment of the agent of the present invention, the substance which suppresses an SMGBP1 activity of the polypeptide is (1) an siRNA designed on the basis of an SMGBP1-specific nucleotide sequence consisting of 19 to 21 nucleotides in a nucleotide sequence of SMGBP1, or (2) an expression vector expressing the siRNA in a subject to be administered.

Further, the present invention relates to an siRNA designed on the basis of an SMGBP1-specific nucleotide sequence consisting of 19 to 21 nucleotides in a nucleotide sequence of SMGBP1.

According to a preferred embodiment of the siRNA of the present invention, the SMGBP1-specific nucleotide sequence is a sequence selected from the group consisting of the sequences of SEQ ID NOS: 9 to 15.

According to another preferred embodiment of the siRNA of the present invention, a double-stranded RNA portion consists of 1st to 19th nucleotides in a sequence of any one of SEQ ID NOS: 16 to 22.

According to another preferred embodiment of the siRNA of the present invention, a sense strand consists of a sequence of any one of SEQ ID NOS: 16 to 22.

Further, the present invention relates to an agent for promoting nonsense-mediated mRNA decay, comprising, as an active ingredient, a substance which promotes an SMGBP1 activity of the above polypeptide.

### BRIEF DESCRIPTION OF THE DRAWINGS

FIG. 1 is an autoradiogram showing the results in which immunoprecipitates of the human HeLa cell extract obtained by using the anti-hSMG-1 antiserum (antiserum C) were phosphorylated.

FIG. 2 is an autoradiogram showing the results in which immunoprecipitates of the human HeLa cell extract obtained by using two anti-hSMG-1 antisera (antiserum C or antiserum L) were phosphorylated.

FIG. 3 is a chromatogram of the human HeLa cell extract with anion exchange column HiTrapQ.

FIG. 4 is a drawing showing the results of Western blotting performed to show the distribution of human SMG-1 in the fractions described in FIG. 3.

FIG. 5 is an autoradiograph showing the results in which immunoprecipitates of the fractions described in FIG. 3 obtained by using the hSMG-1 serum were phosphorylated.

FIG. 6 is a drawing showing the results in which immunoprecipitates of fractions 20 to 31 described in FIG. 3 obtained by using the hSMG-1 serum were phosphorylated, and subjected to Western blotting.

FIG. 7 is a drawing showing the results in which immunoprecipitates of the human HeLa cell extract obtained by using the anti-hSMG-1 IgG (antibody N) were subjected to SDS-PAGE, and stained by silver staining.

FIG. 8 illustrates the primary structure of the translated product of AK026858.1, and the positions of sequences corresponding to AP1, AP2, AP3, AP4, AP7, and AP8.

FIG. 9 is a drawing showing the results in which immunoprecipitates of the human HeLa cell extract obtained by using the anti-hSMG-1 antisera (antiserum N, antiserum L, or antiserum C) were subjected to Western blotting using the anti AK026858.1-translated-product antibody.

FIG. 10 is a Western blot showing the results in which the phosphorylation of hUPF1 in cells transfected with p130-specific siRNAs was analyzed.

FIG. 11 is a graph showing rates of phosphorylated hUPF1 (P-hUPF1) to hUPF1 (P-hUPF1/hUPF1) in cells transfected with p130-specific siRNAs.

### BEST MODE FOR CARRYING OUT THE INVENTION

The present invention will be explained in detail hereinafter.

[1] Polypeptide of the Present Invention

The polypeptide of the present invention includes
(1) a polypeptide consisting of the amino acid sequence of SEQ ID NO: 2;
(2) a polypeptide exhibiting an SMGBP1 activity and comprising the amino acid sequence of SEQ ID NO: 2;
(3) a polypeptide exhibiting an SMGBP1 activity and comprising an amino acid sequence in which one or plural amino acids are deleted, substituted, and/or inserted at one or plural positions in the amino acid sequence of SEQ ID NO: 2 (hereinafter referred to as a functionally equivalent mutant); and
(4) a polypeptide exhibiting an SMGBP1 activity and comprising an amino acid sequence having a 90% or more homology with that of SEQ ID NO: 2, (hereinafter referred to as a homologous polypeptide).

The “polypeptide consisting of the amino acid sequence of SEQ ID NO: 2” as the polypeptide of the present invention is a novel protein, which consists of 991 amino acid residues, and may bind to SMG-1 (particularly human SMG-1) to form a complex. The present inventors named the polypeptide “SMGBP1” (SMG Binding Protein 1) on the basis of the activity of binding to SMG-1.

The term “SMGBP1 activity” as used herein means an activity of binding SMG-1 (particularly human SMG-1), an activity of being phosphorylated by SMG-1, and/or an activity of modifying SMG-1 activities through these activities. Further, “exhibiting an SMGBP1 activity” as used herein means that a polypeptide exhibits at least one of these activities.

In this connection, SMG-1 is phosphatidyl inositol kinase (PIK) related protein kinase (PIKK) that is essential to a process of mRNA surveillance, and has an autophosphorylation activity. Further, SMG-1 has an activity of phosphorylating UPF1/SMG-2 [Sun, X. et al., Proc. Natl. Acad. Sci. USA, 1998, 95, p. 10009-100014; and Bhattacharya, A. et al., Rna, 2000, 6, p. 1226-1235] essential to nonsense-mediated mRNA decay (NMD), and modifies NMD positively [Yamashita, Akio. et al., “GENES & DEVELOPMENT”, (USA), 2001, 15, p. 2215-2228].

A method for confirming whether or not a polypeptide to be tested “exhibits an SMGBP1 activity” is not particularly limited. It may be confirmed, for example, by bringing the test polypeptide into contact with SMG-1 (for example, human SMG-1), and analyzing whether or not a complex of the test polypeptide and SMG-1 is formed.

As a method for bringing the test polypeptide into contact with SMG-1 in the above confirmation method, there may be mentioned, for example,
a method in which the test polypeptide and SMG-1 are separately prepared by genetic engineering techniques, and are brought into contact with each other; or
a method in which a polynucleotide encoding the test polypeptide is introduced into cells capable of producing SMG-1, and the test polypeptide and SMG-1 are produced and brought into contact with each other in the cells.

As a method for analyzing the complex of the test polypeptide and SMG-1 in the above confirmation method, there may be mentioned, for example,
a method in which immunoprecipitation is performed using an antibody against either of two polypeptides, i.e., SMG-1 and the test polypeptide, (for example, anti-SMG-1 antibody), and the resulting immunoprecipitate is analyzed by Western blotting using an antibody against the other polypeptide (for example, anti-test-polypeptide antibody) to confirm the existence of the latter polypeptide [for example, a method described in Example 4 (2)];
a method in which immunoprecipitation is performed using an antibody against either SMG-1 or the test polypeptide (for example, anti-SMG-1 antibody), and the resulting immunoprecipitate is electrophoresed and analyzed by an appropriate detection method (for example, silver staining) to confirm the existence of the other polypeptide (for example, the test polypeptide) [for example, a method described in Example 3 (1)];
a method in which immunoprecipitation is performed using an anti-SMG-1 antibody, and the resulting immunoprecipitate is phosphorylated under the conditions such that phosphorylation by SMG-1 may be carried out to confirm the existence of the phosphorylated test polypeptide [for example, a method described in Example 1(2) to Example 1(4)]; or
   a method in which chromatography is carried out to confirm whether or not the test polypeptide and SMG-1 exist in the same fraction [for example, a method described in Example 2(2) to Example 2(3)].

The “polypeptide comprising the amino acid sequence of SEQ ID NO: 2, and exhibiting an SMGBP1 activity” as the polypeptide of the present invention may be, for example, a fusion polypeptide consisting of an amino acid sequence in which an appropriate marker sequence or the like is added to the N-terminus and/or the C-terminus of the amino acid sequence of SEQ ID NO: 2, and exhibiting an SMG-1 activity.

As the marker sequence in the polypeptide of the present invention, for example, a sequence for easily carrying out confirmation of polypeptide expression, confirmation of intracellular localization thereof, purification thereof, or the like may be used. As the sequence, there may be mentioned, for example, the FLAG tag, the hexa-histidine tag, the hemagglutinin tag, the myc epitope, or the like.

The functionally equivalent mutant of the present invention is not particularly limited, so long as it is a polypeptide comprising an amino acid sequence in which one or plural (preferably 1 to 10, more preferably 1 to 7, most preferably 1 to 5) amino acids, such as one to several amino acids, are deleted, substituted, and/or inserted at one or plural positions of the amino acid sequence of SEQ ID NO: 2, and exhibiting an SMGBP1 activity. Further, an origin of the functionally equivalent mutant is not limited to a human.

The functionally equivalent mutant of the present invention includes, for example, human mutants of the polypeptide (i.e., human SMGBP1) consisting of the amino acid sequence of SEQ ID NO: 2, and functionally equivalent mutants derived from organisms other than human (such as simian, mouse, rat, hamster, or dog). Further, the functionally equivalent mutant of the present invention includes polypeptides prepared using polynucleotides obtained by artificially modifying polynucleotides encoding these native polypeptides (i.e., human mutants or functionally equivalent mutants derived from organisms other than human) or polynucleotides encoding the polypeptide consisting of the amino acid sequence of SEQ ID NO: 2 by genetic engineering techniques.

Human mutants of the polypeptide consisting of the amino acid sequence of SEQ ID NO: 2 or functionally equivalent mutants derived from organisms other than a human may be obtained by those skilled in the art in accordance with the information of a nucleotide sequence (for example, the nucleotide sequence consisting of 16th to 2988th bases in the nucleotide sequence of SEQ ID NO: 1) of a polynucleotide encoding the polypeptide consisting of the amino acid sequence of SEQ ID NO: 2. In this connection, genetic engineering techniques may be generally performed in accordance with known methods (for example, Sambrook, J. et al., “Molecular Cloning-A Laboratory Manual”, Cold Spring Harbor Laboratory, NY, 1989).

For example, an appropriate probe or appropriate primers are designed in accordance with the information of a nucleotide sequence of a polynucleotide encoding the polypeptide consisting of the amino acid sequence of SEQ ID NO: 2. A polymerase chain reaction (PCR) method (Saiki, R. K. et al., Science, 239, 487-491, 1988) or a hybridization method is carried out using a sample (for example, total RNA or an mRNA fraction, a cDNA library, or a phage library) prepared from an organism (for example, a mammal such as human, simian, mouse, rat, hamster, or dog) of interest and the primers or the probe to obtain a polynucleotide encoding the polypeptide. A desired polypeptide may be obtained by expressing the resulting polynucleotide in an appropriate expression system and confirming that the expressed polypeptide exhibits an SMGBP1 activity.

Further, the polypeptide artificially modified by genetic engineering techniques may be obtained by, for example, the following procedure. A polynucleotide encoding the polypeptide may be obtained by a conventional method, for example, site-directed mutagenesis (Mark, D. F. et al., Proc. Natl. Acad. Sci. USA, 81, 5662-5666, 1984). A desired polypeptide may be obtained by expressing the resulting polynucleotide in an appropriate expression system and confirming that the expressed polypeptide exhibits an SMGBP1 activity.

The homologous polypeptide of the present invention is not particularly limited, so long as it comprises an amino acid sequence having a 90% or more homology with the amino acid sequence of SEQ ID NO: 2, and exhibits an SMG-1 activity. The homologous polypeptide of the present invention may comprise an amino acid sequence having preferably a 95% or more homology, more preferably a 98% or more homology, most preferably a 99% or more homology, with respect to the amino acid sequence of SEQ ID NO: 2.

The term “homology” as used herein means a value obtained by BLAST [Basic local alignment search tool; Altschul, S. F. et al., J. Mol. Biol., 215, 403-410, (1990)].

[2] Polynucleotide of the Present Invention

The polynucleotide of the present invention is not particularly limited, so long as it encodes the polypeptide of the present invention. As the polynucleotide of the present invention, there may be mentioned, for example, a polynucleotide comprising the nucleotide sequence consisting of the 16th to 2988th bases in the nucleotide sequence of SEQ ID NO: 1, or a polynucleotide consisting of the nucleotide sequence consisting of the 16th to 2988th bases in the nucleotide sequence of SEQ ID NO: 1. The term “polynucleotide” as used herein includes both DNA and RNA.

A method for producing the polynucleotide of the present invention is not particularly limited, but there may be mentioned, for example, (1) a method using PCR, (2) a method using conventional genetic engineering techniques (i.e., a method for selecting a transformant comprising a desired cDNA from strains transformed with a cDNA library), or (3) a chemical synthesis method. These methods will be explained in this order hereinafter.

In the method using PCR of the item (1), the polynucleotide of the present invention may be produced, for example, by the following procedure.
mRNA is extracted from human cells or tissue capable of producing the polypeptide of the present invention. A pair of primers, between which full-length mRNA corresponding to the polypeptide of the present invention or a partial region of the mRNA is located, is synthesized on the basis of the nucleotide sequence of a polynucleotide encoding the polypeptide of the present invention. Full-length cDNA encoding the polypeptide of the present invention or a part of the cDNA may be obtained by performing a reverse transcriptase-polymerase chain reaction (RT-PCR) using the extracted mRNA as a template.

More particularly, total RNA containing mRNA encoding the polypeptide of the present invention is extracted by a known method from cells or tissue capable of producing the polypeptide of the present invention. As an extraction method, there may be mentioned, for example, a guanidine thiocyanate-hot phenol method, a guanidine thiocyanate-guanidine hydrochloride method, or a guanidine thiocyanate-cesium chloride method. The guanidine thiocyanate-cesium chloride method is preferably used. The cells or tissue capable of producing the polypeptide of the present invention may be identified, for example, by a Northern blotting method using a polynucleotide or a part thereof encoding the polypeptide of the present invention or a Western blotting method using an antibody specific for the polypeptide of the present invention.

Next, the extracted mRNA is purified. Purification of the mRNA may be made in accordance with a conventional method. For example, the mRNA may be purified by adsorption and elution using an oligo (dT)-cellulose column. The mRNA may be further fractionated by, for example, a sucrose density gradient centrifugation, if necessary. Alternatively, commercially available extracted and purified mRNA may be used without carrying out the extraction of the mRNA.

Next, the first-strand cDNA is synthesized by carrying out a reverse transcriptase reaction of the purified mRNA in the presence of a random primer, an oligo dT primer, and/or a custom primer. This synthesis may be carried out in accordance with a conventional method. The resulting first-strand cDNA is subjected to PCR using two primers between which a full-length or a partial region of the polynucleotide of interest is located, thereby amplifying the cDNA of interest. The resulting DNA is fractionated by, for example, an agarose gel electrophoresis. The DNA fragment of interest may be obtained by carrying out a digestion of the DNA with restriction enzymes and subsequent ligation, if necessary.

In the method using conventional genetic engineering techniques of the item (2), the polynucleotide of the present invention may be produced, for example, by the following procedure.

First, single-stranded cDNA is synthesized by using reverse transcriptase from mRNA prepared by the above-mentioned PCR method as a template, and then double-stranded cDNA is synthesized from the single-stranded cDNA. As this method, there may be mentioned, for example, an S1 nuclease method (Efstratiadis, A. et al., Cell, 7, 279-288, 1976), a Land method (Land, H. et al., Nucleic Acids Res., 9, 2251-2266, 1981), an O. Joon Yoo method (Yoo, O. J. et al., Proc. Natl. Acad. Sci. USA, 79, 1049-1053, 1983), and an Okayama-Berg method (Okayama, H. and Berg, P., Mol. Cell. Biol., 2, 161-170, 1982).

Next, a recombinant plasmid comprising the double-stranded cDNA is prepared and introduced into an Escherichia coli strain, such as DH 5α, HB101, or JM109, thereby transforming the strain. A transformant is selected using a drug resistance against, for example, tetracycline, ampicillin, or kanamycin as a marker. When the host cell is E. coli, transformation of the host cell may be carried out, for example, by the method of Hanahan (Hanahan, D. J., Mol. Biol., 166, 557-580, 1983); namely, a method in which the recombinant DNA is added to competent cells prepared in the presence of CaCl₂, MgCl₂, or RbCl. Further, as a vector other than a plasmid, a phage vector such as a lambda system may be used.

As a method for selecting a transformant containing the cDNA of interest from the resulting transformants, various methods such as (i) a method for screening a transformant using a synthetic oligonucleotide probe, (ii) a method for screening a transformant using a probe produced by PCR, (iii) a method for screening a transformant using an antibody against the polypeptide of the present invention, or (iv) a method for screening a transformant using a selective hybridization translation system, may be used.

In the method of the item (i) for screening a transformant using a synthetic oligonucleotide probe, the transformant containing the cDNA of interest may be selected, for example, by the following procedure.

An oligonucleotide which corresponds to the whole or a part of the polypeptide of the present invention is synthesized (in this case, it may be either a nucleotide sequence taking the codon usage into consideration or a plurality of nucleotide sequences as a combination of possible nucleotide sequences, and in the latter case, their numbers can be reduced by including inosine) and, using this oligonucleotide as a probe (labeled with ³²P or ³³P), hybridized with a nitrocellulose filter or a polyamide filter on which DNAs of the transformants are denatured and fixed, to screen and select resulting positive strains.

In the method of the item (ii) for screening a transformant using a probe produced by PCR, the transformant containing the cDNA of interest may be selected, for example, by the following procedure.

Oligonucleotides of a sense primer and an antisense primer corresponding to a part of the polypeptide of the present invention are synthesized, and a DNA fragment encoding the whole or a part of the polypeptide of interest is amplified by carrying out PCR using these primers in combination. As a template DNA used in this method, cDNA synthesized by a reverse transcription reaction from mRNA of cells capable of producing the polypeptide of the present invention, or genomic DNA, may be used. The resulting DNA fragment is labeled with ³²P or ³³P, and a transformant containing the cDNA of interest is selected by carrying out colony hybridization or a plaque hybridization using this fragment as a probe.

In the method of the item (iii) for screening a transformant using an antibody against the polypeptide of the present invention, the transformant containing the cDNA of interest may be selected, for example, by the following procedure.

Polypeptides are produced into a culture supernatant, inside the cells, or on the cell surface of transformants. A transformant containing the cDNA of interest is selected by detecting a strain producing the desired polypeptide using an antibody against the polypeptide of the present invention and a second antibody against the first antibody.

In the method of the item (iv) for screening a transformant using a selective hybridization translation system, the transformant containing the cDNA of interest may be selected, for example, by the following procedure.

First, cDNA obtained from each transformant is blotted on, for example, a nitrocellulose filter and hybridized with mRNA prepared from cells capable of producing the polypeptide of the present invention, and then the mRNA bound to the cDNA is dissociated and recovered. The recovered mRNA is translated into a polypeptide in an appropriate polypeptide translation system, for example, injection into Xenopus oocytes or a cell-free system such as a rabbit reticulocyte lysate or a wheat germ. A transformant containing the cDNA of interest is selected by detecting it with the use of an antibody against the polypeptide of the present invention.

A method for collecting the polynucleotide of the present invention from the resulting transformant of interest can be carried out in accordance with a known method (for example, Sambrook, J. et al., “Molecular Cloning-A Laboratory Manual”, Cold Spring Harbor Laboratory, NY, 1989). For example, it may be carried out by separating a fraction corresponding to the plasmid DNA from cells and cutting out the cDNA region from the plasmid DNA.

In the chemical synthesis method of the item (3), the polynucleotide of the present invention may be produced, for example, by binding DNA fragments produced by a chemical synthesis method. Each DNA can be synthesized using a DNA synthesizer [for example, Oligo 1000M DNA Synthesizer (Beckman) or 394 DNA/RNA Synthesizer (Applied Biosystems)].

Further, the polynucleotide of the present invention may be produced by nucleic acid chemical synthesis in accordance with a conventional method such as a phosphite triester method (Hunkapiller, M. et al., Nature, 10, 105-111, 1984), based on the information on the polypeptide of the present invention. In this connection, codons for each amino acid are known and can be optionally selected and determined by the conventional method, for example, by taking a codon usage of each host to be used into consideration (Crantham, R. et al., Nucleic Acids Res., 9, r43-r74, 1981). Further, a partial modification of codons of these nucleotide sequences can be carried out in accordance with a conventional method, such as site directed mutagenesis which uses a primer comprised of a synthetic oligonucleotide coding for a desired modification (Mark, D. F. et al., Proc. Natl. Acad. Sci. USA, 81, 5662-5666, 1984).

Determination of the DNA sequences obtained by the above-mentioned methods can be carried out by, for example, a Maxam-Gilbert chemical modification method (Maxam, A. M. and Gilbert, W., “Methods in Enzymology”, 65, 499-559, 1980) or a dideoxynucleotide chain termination method (Messing, J. and Vieira, J., Gene, 19, 269-276, 1982).

[3] Expression Vector and Transformant of the Present Invention

An isolated polynucleotide of the present invention is re-integrated into an appropriate vector DNA and a eucaryotic or procaryotic host cell may be transfected by the resulting expression vector. Further, it is possible to express the polynucleotide in a desired host cell, by introducing an appropriate promoter and a sequence related to the gene expression into the vector.

The expression vector of the present invention is not particularly limited, so long as it comprises the polynucleotide of the present invention. As the expression vector, there may be mentioned, for example, an expression vector obtained by introducing the polynucleotide of the present invention into a known expression vector appropriately selected in accordance with a host cell to be used or a cell to be introduced. The expression vector of the present invention includes an expression vector for manufacturing the recombinant polypeptide of the present invention and an expression vector for producing the polypeptide of the present invention in a body by gene therapy.

The transformant of the present invention is not particularly limited, so long as it is transfected with the expression vector or the polynucleotide of the present invention and comprises the polynucleotide of the present invention. The transformant of the present invention may be, for example, a cell in which the polynucleotide is integrated into a chromosome of a host cell, or a cell containing the polynucleotide as an expression vector comprising polynucleotide. Further, the transformant of the present invention may be a cell expressing the polypeptide of the present invention, or a cell not expressing the polypeptide of the present invention. The transformant of the present invention may be obtained by, for example, transfecting a desired host cell with the expression vector of the present invention.

In the eucaryotic host cells, for example, cells of vertebrates, insects, and yeast are included. As the vertebral cell, there may be mentioned, for example, a simian COS cell (Gluzman, Y., Cell, 23, 175-182, 1981), a dihydrofolate reductase defective strain of a Chinese hamster ovary cell (CHO) (Urlaub, G. and Chasin, L. A., Proc. Natl. Acad. Sci. USA, 77, 4216-4220, 1980), a human fetal kidney derived HEK293 cell, a 293-EBNA cell (Invitrogen) obtained by introducing an EBNA-1 gene of Epstein Barr Virus into HEK293 cell, or a human 293T cell (DuBridge, R. B. et al., Mol. Cell. Biol., 7, 379-387, 1987).

As an expression vector for a vertebral cell, a vector containing a promoter positioned upstream of the gene to be expressed, an RNA splicing site, a polyadenylation site, a transcription termination sequence, and the like may be generally used. The vector may further contain a replication origin, if necessary. As the expression vector, there may be mentioned, for example, pSV2dhfr containing an SV40 early promoter (Subramani, S. et al., Mol. Cell. Biol., 1, 854-864, 1981), pEF-BOS containing a human elongation factor promoter (Mizushima, S. and Nagata, S., Nucleic Acids Res., 18, 5322, 1990), or pCEP4 containing a cytomegalovirus promoter (Invitrogen).

When the COS cell is used as the host cell, a vector which has an SV40 replication origin, can perform an autonomous replication in the COS cell, and has a transcription promoter, a transcription termination signal, and an RNA splicing site, may be used as the expression vector. As the vector, there may be mentioned, for example, pME18S (Maruyama, K. and Takebe, Y., Med. Immunol., 20, 27-32, 1990), pEF-BOS (Mizushima, S. and Nagata, S., Nucleic Acids Res., 18, 5322, 1990), or pCDM8 (Seed, B., Nature, 329, 840-842, 1987).

The expression vector may be incorporated into COS cells by, for example, a DEAE-dextran method (Luthman, H. and Magnusson, G., Nucleic Acids Res., 11, 1295-1308, 1983), a calcium phosphate-DNA co-precipitation method (Graham, F. L. and van der Ed, A. J., Virology, 52, 456-457, 1973), a method using a commercially available transfection reagent (for example, FuGENE™6 Transfection Reagent; Boeringer Mannheim), or an electroporation method (Neumann, E. et al., EMBO J., 1, 841-845, 1982).

When the CHO cell is used as the host cell, a transfected cell capable of stably producing the polypeptide of the present invention can be obtained by carrying out co-transfection of an expression vector comprising the polynucleotide encoding the polypeptide of the present invention, together with a vector capable of expressing a neo gene which functions as a G418 resistance marker, such as pRSVneo (Sambrook, J. et al., “Molecular Cloning—A Laboratory Manual”, Cold Spring Harbor Laboratory, NY, 1989) or pSV2-neo (Southern, P. J. and Berg, P., J. Mol. Appl. Genet., 1, 327-341, 1982), and selecting a G418 resistant colony.

As a vector for a gene therapy, a vector generally used (for example, a retrovirus vector, an adenovirus vector, or Sendai virus vector) can be used.

The transformant of the present invention may be cultured in accordance with the conventional method, and the polypeptide of the present invention is produced inside the cells. As a medium to be used in the culturing, a medium commonly used in a desired host cell may be appropriately selected. In the case of the COS cell, for example, a medium such as an RPMI-1640 medium or a Dulbecco's modified Eagle's minimum essential medium (DMEM) may be used, by supplementing it with a serum component such as fetal bovine serum (FBS) if necessary. In the case of the 293-EBNA cell, a medium such as a Dulbecco's modified Eagle's minimum essential medium (DMEM) with a serum component such as fetal bovine serum (FBS) and G418 may be used.

The polypeptide of the present invention produced inside the transformant of the present invention by culturing the transformants may be separated and purified therefrom by various known separation techniques making use of the physical properties, chemical properties and the like of the polypeptide. More particularly, the polypeptide of the present invention may be purified by treating a cell extract containing the polypeptide of the present invention with a commonly used treatment, for example, a treatment with a protein precipitant, ultrafiltration, various liquid chromatography techniques such as molecular sieve chromatography (gel filtration), adsorption chromatography, ion exchange chromatography, affinity chromatography, or high performance liquid chromatography (HPLC), or dialysis, or a combination thereof.

When the polypeptide of the present invention is expressed as a fusion protein with a marker sequence in frame, identification of the expression of the polypeptide of the present invention, purification thereof, or the like may be easily carried out. As the marker sequence, there may be mentioned, for example, a FLAG tag, a hexa-histidine tag, a hemagglutinin tag, or a myc epitope. Further, by inserting a specific amino acid sequence recognized by a protease such as enterokinase, factor Xa, or thrombin between the marker sequence and the polypeptide of the present invention, the marker sequence may be removed by the protease.

[4] Screening Method of the Present Invention

It is possible to screen a substance which modifies (for example, inhibits or promotes) an SMGBP1 activity of the polypeptide according to the present invention, using the polypeptide of the present invention. Human SMGBP1 as the polypeptide of the present invention firmly binds to human SMG-1 that is essential to a process of mRNA surveillance, and thus, it is predicted that human SMGBP1 is closely involved in the modification of a kinase activity, intracellular localization, and the modification of an activity of a surveillance complex. Therefore, a substance modifying an SMGBP1 activity of the polypeptide of the present invention may modify nonsense mediated mRNA decay (NMD), for example, via a modification of an SMG-1 activity, and thus is useful as a candidate substance of an agent for treating and/or preventing a variety of diseases related to NMD. Further, the polypeptide of the present invention per se may be used as a screening tool for screening a substance modifying an SMGBP1 activity of the polypeptide of the present invention, or for screening an agent for treating and/or preventing various diseases related to NMD.

In this connection, NMD means a mechanism which recognizes and specifically degrades a nonsense mutation mRNA in which a codon in the inherent translational region of a gene is changed to a stop codon.

As the diseases related to NMD, there may be mentioned, for example, a disease due to mRNA having one or more premature translation termination codons (PTCs), which should be removed, but are not removed, or a disease caused by one or more PTCs generated by a nonsense mutation.

The disease caused by one or more PTCs generated by a nonsense mutation is not particularly limited, but there may be mentioned, for example, a genetic disease (for example, Duchenne type muscular dystrophy), cancer due to a somatic mutation, or the like. The important point is that, among all diseases caused by genome mutation, almost all diseases “due to one or more PTCs by a nonsense mutation” are included in such diseases.

One-quarter of the diseases caused by genome mutations are said to have the termination codon in the middle of a specific gene. The reasons for these diseases are that the protein consisting of the full-length polypeptide inherently encoded by the gene is not expressed, and that, due to the presence of the NMD mechanism, almost no protein fragments consisting of the N-terminal partial fragments of the full length polypeptide inherently encoded by the gene are expressed. However, even if there is a termination codon in the middle of the gene, and even if in the state of a protein fragment, there are cases of activity of the same extent as that of a full length polypeptide or the minimum necessary level, depending on the type of the gene or the position of the termination codon. In this case, if it were possible to inhibit the NMD mechanism, it would become possible to express a protein fragment having an effective activity, and thus it is theoretically predicted that at least part of a disease due to the presence of a termination codon in the middle of a specific gene, that is, a disease due to nonsense mutation of a specific gene, can be alleviated. However, no technique for a specific suppression of NMD has been found in the past.

Among the substances which are selected by the screening method of the present invention and modify an SMGBP1 activity of the polypeptide of the present invention, substances which can inhibit or suppress an SMG-1 activity via a modification of the SMGBP1 activity, may be expected to specifically inhibit or suppress NMD, and thus are useful as an active ingredient of a new type of agent for treatment and/or prevention, which can alleviate gene mutations for at least part of all sorts of diseases due to a nonsense mutation of a specific gene.

Alternatively, among the substances which are selected by the screening method of the present invention and modify an SMGBP1 activity of the polypeptide of the present invention, substances which can promote an SMG-1 activity via a modification of the SMGBP1 activity, may promote NMD, and thus are useful as an active ingredient of an agent for promoting NMD, or an agent for treating and/or preventing a disease due to mRNA having one or more PTCs, which should be removed, but are not removed.

Substances to be tested which may be applied to the screening method of the present invention are not particularly limited, but there may be mentioned, for example, various known compounds (including peptides) registered in chemical files, compounds obtained by combinatorial chemistry techniques (Terrett, N. K. et al., Tetrahedron, 51, 8135-8137, 1995) or conventional synthesis techniques, or random peptides prepared by employing a phage display method (Felici, F. et al., J. Mol. Biol., 222, 301-310, 1991) or the like. In addition, culture supernatants of microorganisms, natural components derived from plants or marine organisms, or animal tissue extracts may be used as the test substances for screening. Further, compounds (including peptides) obtained by chemically or biologically modifying compounds (including peptides) selected by the screening method of the present invention may be used.

The screening method of the present invention comprises the steps of:
(1) bringing the polypeptide of the present invention into contact with SMG-1 (for example, human SMG-1) and a substance to be tested under the conditions such that the polypeptide exhibits an SMGBP1 activity in the absence of the substance to be tested (hereinafter referred to as a contact step), and
(2) analyzing whether or not the polypeptide exhibits the SMGBP1 activity (hereinafter referred to as an analyzing step).

In the above contact step, the method for bringing the polypeptide of the present invention into contact with SMG-1 and a substance to be tested is not particularly limited, so long as these can be brought into contact with each other under the conditions such that the polypeptide of the present invention may exhibit an SMGBP1 activity (i.e., the polypeptide of the present invention may bind to SMG-1) in the absence of the test substance. As the contacting method, there may be mentioned, for example,
a method in which the polypeptide and SMG-1 are separately prepared by genetic engineering techniques, and the polypeptide, SMG-1, and the test substance are brought into contact with each other; or
a method in which a polynucleotide encoding the polypeptide is introduced into cells capable of producing SMG-1, the polypeptide and SMG-1 are produced in the cell in the presence of the test substance, and the polypeptide, SMG-1, and the test substance are brought into contact with each other.

In the above analyzing step, it is analyzed whether or not the polypeptide of the present invention exhibits an SMGBP1 activity. More particularly, for example, it is analyzed whether or not the complex of SMG-1 and the polypeptide of the present invention is formed.

In the analyzing step, when the complex of SMG-1 and the polypeptide of the present invention is not formed, it may be judged that the substance to be tested inhibits or suppresses an SMGBP1 activity of the polypeptide of the present invention.

In the analyzing step, as the method for analyzing the complex of SMG-1 and the polypeptide of the present invention, there may be mentioned, for example, a method in which immunoprecipitation is performed using an antibody against either of two polypeptides, i.e., SMG-1 and the polypeptide of the present invention, (for example, anti-SMG-1 antibody), and the resulting immunoprecipitate is analyzed by Western blotting using an antibody against the other polypeptide (for example, antibody against the polypeptide of the present invention) to confirm the existence of the latter polypeptide [hereinafter referred to as the method using immunoprecipitation and Western blotting; for example, a method described in Example 4 (2)]; a method in which immunoprecipitation is performed using an antibody against either SMG-1 or the polypeptide of the present invention (for example, anti-SMG-1 antibody), and the resulting immunoprecipitate is electrophoresed and analyzed by an appropriate detection method (for example, silver staining) to confirm the existence of the other polypeptide (for example, the polypeptide of the present invention) [hereinafter referred to as the method using immunoprecipitation and silver staining; for example, a method described in Example 3(1)];
a method in which immunoprecipitation is performed using an anti-SMG-1 antibody, and the resulting immunoprecipitate is phosphorylated under the conditions such that phosphorylation by SMG-1 may be carried out to confirm the existence of the phosphorylated polypeptide of the present invention [hereinafter referred to as the method using immunoprecipitation and phosphorylation; for example, a method described in Example 1(2) to Example 1(4)]; or a method in which chromatography is carried out to confirm whether or not the polypeptide of the present invention and SMG-1 exist in the same fraction [hereinafter referred to as the fraction method; for example, a method described in Example 2(2) to Example 2(3)].

When the above method using immunoprecipitation and Western blotting is used in the analyzing step, immunoprecipitation is carried out using an antibody against either of two polypeptides, i.e., SMG-1 and the polypeptide of the present invention, (for example, anti-SMG-1 antibody), and the resulting immunoprecipitate is analyzed by Western blotting using an antibody against the other polypeptide (for example, antibody against the polypeptide of the present invention). As a result of the Western blotting, when the latter polypeptide (for example, the polypeptide of the present invention) is detected, it may be judged that the complex of SMG-1 and the polypeptide of the present invention is formed (i.e. the polypeptide of the present invention exhibits an SMGBP1 activity). On the other hand, when the latter polypeptide (for example, the polypeptide of the present invention) is not detected, it may be judged that the complex of SMG-1 and the polypeptide of the present invention is not formed (i.e. the polypeptide of the present invention does not exhibit an SMGBP1 activity), and thus, it may be determined that the substance to be tested is a substance which inhibits or suppresses an SMGBP1 activity of the polypeptide of the present invention. Similarly, it may be determined whether or not a substance to be tested is a substance which promotes an SMGBP1 activity of the polypeptide of the present invention.

When the above method using immunoprecipitation and silver staining is used in the analyzing step, immunoprecipitation is performed using an antibody against either SMG-1 or the polypeptide of the present invention (for example, anti-SMG-1 antibody), and the resulting immunoprecipitate is electrophoresed and analyzed by an appropriate detection method (for example, silver staining) to confirm the existence of the other polypeptide (for example, the polypeptide of the present invention). When the latter polypeptide (for example, the polypeptide of the present invention) is detected, it may be judged that the complex of SMG-1 and the polypeptide of the present invention is formed (i.e. the polypeptide of the present invention exhibits an SMGBP1 activity). On the other hand, when the latter polypeptide (for example, the polypeptide of the present invention) is not detected, it may be judged that the complex of SMG-1 and the polypeptide of the present invention is not formed (i.e. the polypeptide of the present invention does not exhibit an SMGBP1 activity), and thus, it may be determined that the substance to be tested is a substance which inhibits or suppresses an SMGBP1 activity of the polypeptide of the present invention. Similarly, it may be determined whether or not a substance to be tested is a substance which promotes an SMGBP1 activity of the polypeptide of the present invention.

When the above method using immunoprecipitation and phosphorylation is used in the analyzing step, immunoprecipitation is performed using an anti-SMG-1 antibody, and the resulting immunoprecipitate is phosphorylated under the conditions such that phosphorylation by SMG-1 may be carried out to confirm the existence of the phosphorylated polypeptide of the present invention. When the polypeptide of the present invention is detected, it may be judged that the complex of SMG-1 and the polypeptide of the present invention is formed (i.e. the polypeptide of the present invention exhibits an SMGBP1 activity). On the other hand, when the polypeptide of the present invention is not detected, it may be judged that the complex of SMG-1 and the polypeptide of the present invention is not formed (i.e. the polypeptide of the present invention does not exhibit an SMGBP1 activity), and thus, it may be determined that the substance to be tested is a substance which inhibits or suppresses an SMGBP1 activity of the polypeptide of the present invention. Similarly, it may be determined whether or not a substance to be tested is a substance which promotes an SMGBP1 activity of the polypeptide of the present invention.

When the above fraction method is used in the analyzing step, chromatography is carried out to confirm whether or not the polypeptide of the present invention and SMG-1 exist in the same fraction. When SMG-1 and the polypeptide of the present invention exist in the same fraction, it may be judged that the complex of SMG-1 and the polypeptide of the present invention is formed (i.e. the polypeptide of the present invention exhibits an SMGBP1 activity). On the other hand, when the polypeptide of the present invention and SMG-1 do not exist in the same fraction, it may be judged that the complex of SMG-1 and the polypeptide of the present invention is not formed (i.e. the polypeptide of the present invention does not exhibit an SMGBP1 activity), and thus, it may be determined that the substance to be tested is a substance which inhibits or suppresses an SMGBP1 activity of the polypeptide of the present invention. Similarly, it may be determined that a substance to be tested is a substance which promotes an SMGBP1 activity of the polypeptide of the present invention.

[5] Pharmaceutical Composition of the Present Invention

Among the substances modifying an SMGBP1 activity, which may be selected by the screening method of the present invention, substances which suppress an SMG-1 activity via a modification of an SMGBP1 activity may suppress NMD, and thus are useful as a candidate substance of an agent for treating and/or preventing a disease caused by one or more PTCs generated by a nonsense mutation. The substance modifying an SMGBP1 activity can be administered to a subject (for example, an animal, preferably a mammal, particularly a human) in need of suppressing NMD or in need of treatment and/or prevention of a disease caused by one or more PTCs generated by a nonsense mutation, with or without, but preferably with, a pharmaceutically or veterinarily acceptable ordinary carrier or diluent, in an amount effective therefor.

As the substances which suppress an SMG-1 activity via a modification of an SMGBP1 activity, there may be mentioned, for example, substances suppressing an SMGBP1 activity. The substances suppressing an SMGBP1 activity include, for example, an siRNA against SMGBP1, or an expression vector expressing the siRNA in a subject to be administered.

The siRNAs against SMGBP1 and the expression vectors expressing the same are novel compounds and are included in the present invention.

The siRNA of the present invention is composed of a double-stranded RNA portion, and preferably one or two overhangs at the 3′ termini of sense and/or antisense strands. The overhang is composed of one or two arbitrary nucleotides (for example, ribonucleotides or deoxyribonucleotides), preferably two nucleotides. The nucleotide forming the overhang is preferably a nucleotide complementary to a target nucleotide sequence of SMGBP1, but a nucleotide not complementary thereto exhibits a sufficient RNAi effect. Further, an siRNA with no overhangs also exhibits the RNAi effect.

The sense or antisense strand in the siRNA of the present invention may consist of 21 to 23 nucleotides. The sense and antisense strands may consist of equal numbers of nucleotides or different numbers of nucleotides, preferably equal numbers thereof.

As ribonucleotides forming the 3′ overhang of the siRNA, there may be mentioned, for example, U (uridine), A (adenosine), G (guanosine), or C (cytidine). As deoxyribonucleotides forming the 3′ overhang of the siRNA, there may be mentioned, for example, dT (deoxythymidine), dA (deoxyadenosine), dG (deoxyguanosine), or dC (deoxycytidine).

As the 3′ overhang(s) of the sense and/or antisense strand(s), one or two overhangs consisting of one or two U(s) or dT(s) added to the 3′ end(s) of the sense and/or antisense strand(s), independently, are preferable; a combination of an overhang consisting of one or two U(s) or dT(s) added to the 3′ end of the sense strand and an overhang consisting of one or two U(s) or dT(s) added to the 3′ end of the antisense strand is more preferable; and a combination of an overhang consisting of two Us or dTs added to the 3′ end of the sense strand and an overhang consisting of two Us or dTs added to the 3′ end of the antisense strand is most preferable.

The siRNA of the present invention is not particularly limited, so long as it can specifically suppress an expression of SMGBP1 [preferably mammal (for example, human, simian, mouse, rat, hamster, or dog) SMGBP1, more preferably human SMGBP1 (hSMGBP1)], and is designed on the basis of an SMGBP1-specific nucleotide sequence consisting of, preferably 19 to 21 nucleotides, more preferably 19 to 21 nucleotides subsequent to NA (N=arbitrary nucleotides), most preferably 19 to 21 nucleotides subsequent to AA. The siRNA of the present invention can be designed in accordance with conventional methods, for example, Natasha J. Caplen, Susan Parrish, Farhad Imani, Andrew Fire, and Richard A. Morgan. Specific inhibition of gene expression by small double-stranded RNAs in invertebrate and vertebrate systems. PNAS (PROCEEDINGS OF THE NATIONAL ACADEMY OF SCIENCES USA) vol. 98 no. 17 9742-9747, 2001 (August 14); Sayda M. Elbashir, Jens Harborth, Winfried Lendeckel, Abdullah Yalcin, Klaus Weber & Thomas Tuschl. Duplexes of 21-nucleotide RNAs mediate RNA interference in cultured mammalian cells. NATURE VOL 411, 494-498, 2001. (24 MAY); or Jens Harborth, Sayda M. Elbashir, Kim Bechert, Thomas Tuschl and Klaus Weber. Identification of essential genes in cultured mammalian cells using small interfering RNAs. JOURNAL OF CELL SCIENCE 114 (24) 4557-4565, 2001.

For example, initially, NA (preferably AA) which first appears 50 or more nucleotides (nt) downstream from the initiation codon of SMGBP1 is selected (to avoid binding sites of transcription factors). A 19 to 21-nt sequence subsequent to the NA is recorded, and a GC content of 21 to 23-nt sequence including the NA is calculated to confirm that the GC content is around 50%. When the GC content is lower than 30% or more than 70%, or when plural candidates for siRNA are needed, next NA is selected downstream from the first NA, and the GC content thereof is calculated. It is confirmed that the obtained candidates are specific to SMGBP1 by carrying a database search such as a BLAST (Basic local alignment search tool; J. Mol. Biol., 215, 403-410, 1990) search in NCBI (National Center for Biotechnology Information). Although “NA” is selected in the above procedure, any two nucleotides (NN) can be used instead of NA.

When a DNA nucleotide sequence for designing the siRNA is obtained, an siRNA based on the sequence can be designed. For example, an siRNA in which the double-stranded RNA portion consists of a nucleotide sequence obtained by converting the DNA nucleotide sequence into the corresponding RNA nucleotide sequence can be designed. In this connection, “converting a DNA nucleotide sequence to the corresponding RNA nucleotide sequence” as used herein means that dT in the DNA nucleotide sequence is converted into U, and the other nucleotides, i.e., dA, dG, and dC are converted into A, G, and C, respectively.

As the SMGBP1-specific nucleotide sequence, there may be mentioned, for example, nucleotide sequences of SEQ ID NOS: 9 to 15, preferably nucleotide sequences of SEQ ID NOS: 9, 10, 11, 12, or 15, more preferably nucleotide sequences of SEQ ID NOS: 10, 12, or 15, most preferably nucleotide sequence of SEQ ID NO: 15.

As the siRNA of the present invention, there may be mentioned, for example, siRNAs designed on the basis of any one of the nucleotide sequences of SEQ ID NOS: 9 to 15. Such siRNAs include siRNAs in which the double-stranded RNA portion consists of the RNA sequence corresponding to the nucleotide sequences of SEQ ID NOS: 9 to 15. The siRNAs include siRNAs with the 3′ overhang(s) of the sense and/or antisense strand(s) and siRNAs with no 3′ overhang.

As the siRNA of the present invention, there may be mentioned, for example, the siRNA consisting of the nucleotide sequence of any one of SEQ ID NOS: 16 to 22. The siRNAs consisting of the nucleotide sequence of SEQ ID NOS: 16, 17, 18, 19, or 22 are preferable, the siRNAs consisting of the nucleotide sequence of SEQ ID NOS: 17, 19, or 22 are more preferable, and the siRNA consisting of the nucleotide sequence of SEQ ID NO: 22 is most preferable.

The siRNA of the present invention may be prepared in accordance with conventional methods, such as a chemical synthesis method or genetically engineering techniques.

When the expression vector expressing the siRNA in a subject to be administered is used as an active ingredient in the pharmaceutical composition of the present invention, the siRNA of the present invention can be expressed in the subject by administering to the subject the expression vector constructed by inserting a DNA encoding the siRNA into an appropriate vector for a gene therapy [For example, an adenovirus vector, an adeno-associated virus (AAM) vector, or lentivirus vector].

On the other hand, among the substances modifying an SMGBP1 activity, which may be selected by the screening method of the present invention, substances which promote an SMG-1 activity of the polypeptide of the present invention via a modification of an SMGBP1 activity, can promote NMD, and thus are useful as a candidate substance of an agent for treating and/or preventing a disease due to mRNA having one or more PTCs, which should be removed, but are not removed. The substance modifying an SMGBP1 activity can be administered to a subject (for example, an animal, preferably a mammal, particularly a human) in need of promoting NMD or in need of treating and/or preventing a disease due to mRNA having one or more PTCs, which should be removed, but are not removed, with or without, but preferably with, a pharmaceutically or veterinarily acceptable ordinary carrier or diluent, in an amount effective therefor.

As the substances which promote an SMG-1 activity via a modification of an SMGBP1 activity, there may be mentioned, for example, substances promoting an SMGBP1 activity.

The formulation of the pharmaceutical composition of the present invention is not particularly limited to, but may be, for example, oral medicines, such as powders, fine particles, granules, tablets, capsules, suspensions, emulsions, syrups, extracts or pills, or parenteral medicines, such as injections, liquids for external use, ointments, suppositories, creams for topical application, or eye lotions.

The oral medicines may be prepared by an ordinary method using, for example, fillers, binders, disintegrating agents, surfactants, lubricants, flowability-enhancers, diluting agents, preservatives, coloring agents, perfumes, tasting agents, stabilizers, humectants, antiseptics, antioxidants or the like, such as gelatin, sodium alginate, starch, corn starch, saccharose, lactose, glucose, mannitol, carboxylmethylcellulose, dextrin, polyvinyl pyrrolidone, crystalline cellulose, soybean lecithin, sucrose, fatty acid esters, talc, magnesium stearate, polyethylene glycol, magnesium silicate, silicic anhydride, or synthetic aluminum silicate.

The parenteral administration may be, for example, an injection such as a subcutaneous or intravenous injection, or a per rectum administration. Of the parenteral formulations, an injection is preferably used.

When the injections are prepared, for example, water-soluble solvents, such as physiological saline or Ringer's solution, water-insoluble solvents, such as plant oil or fatty acid ester, agents for rendering isotonic, such as glucose or sodium chloride, solubilizing agents, stabilizing agents, antiseptics, suspending agents, or emulsifying agents may be optionally used, in addition to the active ingredient.

The pharmaceutical composition of the present invention may be administered in the form of a sustained release preparation using sustained release polymers. For example, the pharmaceutical composition of the present invention may be incorporated to a pellet made of ethylenevinyl acetate polymers, and the pellet may be surgically implanted in a tissue to be treated.

The pharmaceutical composition of the present invention may contain the active ingredient in an amount of, but is by no means limited to, 0.01 to 99% by weight, preferably 0.1 to 80% by weight.

A dose of the pharmaceutical composition of the present invention is not particularly limited, but may be determined dependent upon, for example, the kind of the active ingredient, the kind of disease, the age, sex, body weight, or symptoms of the subject, a method of administration, or the like. The pharmaceutical composition of the present invention may be orally or parenterally administered.

The pharmaceutical composition of the present invention may be administered as a medicament or in various forms, for example, eatable or drinkable products, such as functional foods or health foods, or feeds.

[6] Antibody of the Present Invention

An antibody, such as a polyclonal antibody or a monoclonal antibody, which reacts with the polypeptide of the present invention may be obtained by directly administering the polypeptide of the present invention or a fragment thereof to various animals. Alternatively, it may be obtained by a DNA vaccine method (Raz, E. et al., Proc. Natl. Acad. Sci. USA, 91, 9519-9523, 1994; or Donnelly, J. J. et al., J. Infect. Dis., 173, 314-320, 1996), using a plasmid into which a polynucleotide encoding the polypeptide of the present invention is inserted.

The polyclonal antibody may be produced from a serum or eggs of an animal such as a rabbit, a rat, a goat, or a chicken, in which the animal is immunized and sensitized by the polypeptide of the present invention or a fragment thereof emulsified in an appropriate adjuvant (for example, Freund's complete adjuvant) by intraperitoneal, subcutaneous, or intravenous administration. The polyclonal antibody may be separated and purified from the resulting serum or eggs in accordance with conventional methods for polypeptide isolation and purification. Examples of the separation and purification methods include, for example, centrifugal separation, dialysis, salting-out with ammonium sulfate, or a chromatographic technique using such as DEAE-cellulose, hydroxyapatite, protein A agarose, and the like.

The monoclonal antibody may be easily produced by those skilled in the art, according to, for example, a cell fusion method of Kohler and Milstein (Kohler, G. and Milstein, C., Nature, 256, 495-497, 1975).

A mouse is immunized intraperitoneally, subcutaneously, or intravenously several times at an interval of a few weeks by a repeated inoculation of emulsions in which the polypeptide of the present invention or a fragment thereof is emulsified into a suitable adjuvant such as Freund's complete adjuvant. Spleen cells are removed after the final immunization, and then fused with myeloma cells to prepare hybridomas.

As a myeloma cell for obtaining a hybridoma, a myeloma cell having a marker such as a deficiency in hypoxanthine-guanine phosphoribosyltransferase or thymidine kinase (for example, mouse myeloma cell line P3X63Ag8.U1) may be used. As a fusing agent, polyethylene glycol may be used. As a medium for preparation of hybridomas, for example, a commonly used medium such as an Eagle's minimum essential medium, a Dulbecco's modified minimum essential medium, or an RPMI-1640 medium may be used by adding properly 10 to 30% of a fetal bovine serum. The fused strains may be selected by a HAT selection method. A culture supernatant of the hybridomas is screened by a well-known method such as an ELISA method or an immunohistological method, to select hybridoma clones secreting the antibody of interest. The monoclonality of the selected hybridoma is guaranteed by repeating subcloning by a limiting dilution method. Antibodies in an amount which may be purified are produced by culturing the resulting hybridomas in a medium for 2 to 4 days, or in the peritoneal cavity of a pristane-pretreated BALB/c strain mouse for 10 to 20 days.

The resulting monoclonal antibodies in the culture supernatant or the ascites may be separated and purified by conventional polypeptide isolation and purification methods. Examples of the separation and purification methods include, for example, centrifugal separation, dialysis, salting-out with ammonium sulfate, or chromatographic technique using such as DEAE-cellulose, hydroxyapatite, protein A agarose, and the like.

Further, the monoclonal antibodies or the antibody fragments containing a part thereof may be produced by inserting the whole or a part of a gene encoding the monoclonal antibody into an expression vector and introducing the resulting expression vector into appropriate host cells (such as E. coli, yeast, or animal cells).

Antibody fragments comprising an active part of the antibody such as F(ab′)₂, Fab, Fab′, or Fv may be obtained by a conventional method, for example, by digesting the separated and purified antibodies (including polyclonal antibodies and monoclonal antibodies) with a protease such as pepsin or papain, and separating and purifying the resulting fragments by standard polypeptide isolation and purification methods.

Further, an antibody which reacts to the polypeptide of the present invention may be obtained in a form of single chain Fv or Fab in accordance with a method of Clackson et al. or a method of Zebedee et al. (Clackson, T. et al., Nature, 352, 624-628, 1991; or Zebedee, S. et al., Proc. Natl. Acad. Sci. USA, 89, 3175-3179, 1992). Furthermore, a humanized antibody may be obtained by immunizing a transgenic mouse in which mouse antibody genes are substituted with human antibody genes (Lonberg, N. et al., Nature, 368, 856-859, 1994).

[7] Knockout Non-Human Animal and Cell of the Present Invention

The knockout non-human animal of the present invention is not particularly limited, so long as the expression of the gene encoding the polypeptide of the present invention is partially or completely suppressed. It may be prepared by a method known per se.

For example, by using a recombinant vector containing the polynucleotide of the present invention, and embryonic stem cells of the target non-human animal, such as cow, sheep, goat, pig, horse, mouse, or chicken, the gene encoding the polypeptide of the present invention on the chromosomes thereof is deactivated by a known homologous recombinant technique [for example, Nature, 326, 6110, 295 (1987); or Cell, 51, 3, 503 (1987)], or is replaced with any sequence [for example, Nature, 350, 6315, 243 (1991)], to prepare mutant clones. By using the mutant clones of the embryonic stem cells, a chimeric individual consisting of embryonic stem cell clones and normal cells may be prepared by a technique such as an aggregation chimera method or an injection chimera method into blastocyst in fertilized eggs of the animal. By combining the chimera individual and a normal individual, it is possible to obtain an individual having any mutation in the gene encoding the polypeptide of the present invention located on the chromosomes of cells of the entire body. By further combining the individuals, it is possible to obtain, from homozygous individuals with the mutation in both homologous chromosomes, a knockout non-human animal as an individual in which the expression of the gene encoding the polypeptide of the present invention is partially or completely suppressed.

Further, the cell of the present invention in which the expression of the gene encoding the polypeptide of the present invention is partially or completely suppressed, may be obtained by the above knockout non-human animal.

Furthermore, for the desired cell (for example, a human fatal kidney derived HEK293 cell, a 293-EBNA cell obtained by introducing the EBNA-1 gene of Epstein Barr Virus into the HEK293 cell, or a human 293T cell), the expression of intracellular endogenous SMGBP1 can be suppressed by an RNA interference method (for example, Nature, 411, 494-498, 2002). The present invention also includes the cells.

Further, by introducing mutation into any site of a gene encoding the polypeptide of the present invention on the chromosome, it is also possible to produce a knockout non-human animal. For example, by substituting, deleting, and/or inserting one or more bases with respect to a translation region of the gene encoding the polypeptide of the present invention on the chromosome, it is possible to modify the activity of the gene product.

Further, by introducing a similar mutation in the expression control region, it is also possible to modify, for example, the degree of expression, period of expression, and/or a tissue specificity. Further, by the combination with the Cre-loxP system, it is possible to control, for example, the period of expression, location of expression, and/or the amount of expression, more directly. As such examples, an example in which, using a promoter expressed at a specific region of the brain, the target gene was deleted at only the specific region [Cell, 87, 7, 1317, 1996)], or an example in which, using an adenovirus expressing Cre, the target gene was deleted from the specific organ at the desired period [Science, 278, 5335 (1997)] are known.

Therefore, even for a gene encoding the polypeptide of the present invention on the chromosome, it is possible to control expression at any period or tissue as described above. Further, it is possible to prepare a knockout non-human animal having any insertion, deletion, and/or substitution at the translation region or expression control region. A knockout non-human animal can induce the symptoms of various diseases derived from the polypeptide of the present invention at any period, to any degree, and/or at any location. As described above, the knockout non-human animal of the present invention becomes an extremely useful animal model in the treatment or prevention of various diseases derived from the polypeptide of the present invention.

Further, the knockout non-human animal of the present invention can be used to establish a model animal of a disease due to a gene different from the gene encoding the polypeptide of the present invention. For example, one of the knockout non-human animals of the present invention, that is, an SMGBP1 knockout mouse, and various lines of (apparent) normal mice can be combined. When the normal mouse contains a mutant gene having one or more PTCs, in a mouse obtained by the combination (for example, a homozygous individual having the SMGBP1 mutation in both of the homologous chromosomes or a heterozygous individual having the SMGBP1 mutation in one of the homologous chromosomes), NMD activity is expected to be affected, for example, when NMD is suppressed, and thus the mRNA derived from the mutant gene increases. As a result, for a certain mutant gene, sometimes hidden symptoms surface and some sort of disease appears. It is possible to establish a new disease model mouse.

### EXAMPLES

The present invention now will be further illustrated by, but is by no means limited to, the following Examples.

### Example 1

### Detection of hSMG-1 Binding Protein Phosphorylated Dependently on the Enzymatic Activity of Human SMG-1 (hSMG-1)

(1) Cultivation and Collection of Cells and Preparation of Cell Extract

Human HeLa cells (ATCC: CCL-2) were cultured to 10⁷ cells in accordance with a conventional method. Cells in a culture dish were washed with ice-cold phosphate-buffered saline (PBS), scraped with a cell scraper, and collected by centrifugation. After removing PBS, 3 mL of an F-lysis solution [20 mmol/L Tris-HCl (pH7.5), 0.25 mol/L sucrose, 20 mmol/L β-mercaptoethanol, 1.2 mmol/L EGTA, 1 mmol/L sodium orthovanadate, 1 mmol/L sodium fluoride, 1 mmol/L sodium pyrophosphate, 150 mmol/L sodium chloride, 1% Triton X-100, and 0.5% Nonidet P40] supplemented with protease inhibitors [1 mmol/L phenylmethylsulfonyl fluoride (PMSF), 10 μg/mL aprotinin, and 10 μg/mL leupeptin] was added and thoroughly stirred. Further, the whole was sonicated to completely disrupt the cells. The resultant was centrifuged at 15000 r.p.m. for 15 minutes to collect the supernatant as a cell extract.

(2) Immunoprecipitation

In this example, antiserum C [Yamashita, Akio, et al, “GENES & DEVELOPMENT”, (USA), 2001, 15, p. 2215-2228] was used as an anti-hSMG-1 serum. Antiserum C was obtained by using as an immunogen a C-terminal fragment [corresponding to the sequence consisting of the 3076th to 3542nd amino acid residues in the amino acid sequence of hSMG-1 (3657 amino acids)] of hSMG-1, which was expressed in E. coli as a protein (molecular weight=approximately 70 kDa) fused to glutathione S-transferase (GST).

To 25 μL of protein A Sepharose particles (Amersham Pharmacia), on which 10 μL of the anti-hSMG-1 serum (antiserum C) or the preimmune serum thereof (pre) was previously adsorbed, 1 mL of the cell extract (centrifugation supernatant) obtained in Example 1(1) was added and stirred at 4° C. for 2 hours. After the whole was centrifuged to remove the supernatant, washing solution A (the F-lysis solution supplemented with 0.25 mol/L lithium chloride) was newly added and stirred gently for a short time, and the supernatant was removed by recentrifugation. The washing step was further repeated six times to remove non-specific adsorbates.

(3) Phosphorylation

The resulting protein A Sepharose particles were washed with a 1×kinase reaction solution [10 mmol/L Hepes (pH7.5), 50 mmol/L sodium chloride, 50 mmol/L β-glycerophosphoric acid, and 1 mmol/L dithiothreitol (DTT)] once. To the particles, 25 μL of a 2×kinase reaction solution supplemented with divalent metal ion(s) (20 mmol/L MgCl₂ or 20 mmol/L MnCl₂, or both) and/or 1 μmol/L wortmannin [an inhibitor against phosphatidyl inositol 3-kinase (PI3-kinase)-related protein kinase] was added and stirred a little, and then the whole was allowed to stand on ice for 20 minutes. Further, 25 mL of starting solution (5 μCi [γ-³²P]ATP and 10 μmol/L ATP) was added and stirred a little to carry out a reaction at 30° C. for 15 minutes. The reaction was stopped by adding 50 μL of a 2×SDS sample buffer [200 mmol/L Tris-HCl (pH6.8), 4% sodium dodecyl sulfate (SDS), 12% β-mercaptoethanol, 20% glycerol, and 0.02% bromophenol blue], and the whole was incubated at 100° C. for 4 minutes to denature proteins. Each supernatant thereof was used in the following analysis.

(4) Analysis of Phosphorylated Proteins

To analyze phosphorylated proteins having various molecular weights from a high molecular weight to a low molecular weight with a high resolution, samples to be analyzed were electrophoresed by SDS-polyacrylamid gel electrophoresis (SDS-PAGE) using two different gels having a gel concentration of 6% or 12.5%. After the electrophoresis, the gels were dried, and phosphorylated bands were visualized by autoradiography in accordance with conventional methods.

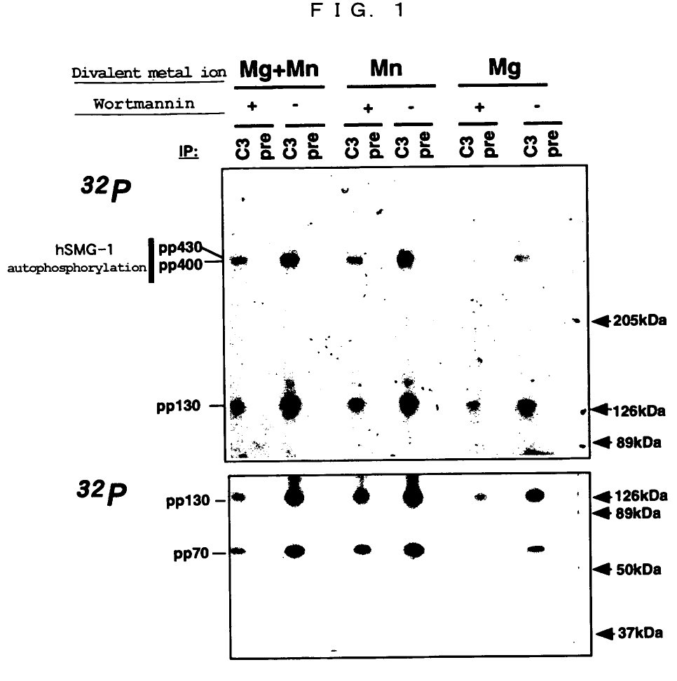

The results are shown in FIG. 1. In FIG. 1, the symbol “³²p” indicates the results obtained by autoradiography. The symbols “+” and “−” indicate the results obtained by performing phosphorylation in the presence of wortmannin, and in the absence thereof, respectively. The symbol “IP” means immunoprecipitation, and the symbols “C3” and “pre” indicate the results obtained by immunoprecipitation using antiserum C and the preimmune serum thereof, respectively. Each molecular weight (for example, “205 kDa”) and each arrow shown at the right of the electrophoretic pattern show the molecular weight of each protein used as molecular markers, and the electrophoresed position thereof, respectively. The symbols “pp430”, “pp400”, “pp130”, and “pp70” shown at the left of the electrophoretic pattern indicate that each protein having a molecular weight of the number in each symbol is phosphorylated.

As shown in FIG. 1, phosphorylated bands were detected at the positions of 400 kDa and 430 kDa. It is supported, from the facts described below, that these bands are autophosphorylated hSMG-1. First, the positions of the two phosphorylated bands accorded with the molecular weights of hSMG-1 detected by Western blotting. Further, the two bands were not detected from the immunoprecipitate precipitated with the preimmune serum. Furthermore, the phosphorylated bands were remarkably decreased in the presence of wortmannin, which was an inhibitor against PI3-kinase-related protein kinases including hSMG-1. Furthermore, the 430 kDa protein and the 400 kDa protein were phosphorylated more strongly when Mn was added, in comparison with the addition of Mg. In this connection, it is known that PI3-kinase-related protein kinases require a divalent metal ion to show the activities (including the autophosphorylation activity) thereof, and prefer Mg to Mn as the divalent metal ion.

In this example, two proteins, that is, a protein having a molecular weight of 130 kDa (p130) and a protein having a molecular weight of 70 kDa (p70), were detected as phosphorylated proteins. The patterns of phosphorylation thereof accorded exactly with those of autophosphorylation of hSMG-1. The results indicate the possibility that the two proteins are binding proteins which may be phosphorylated by the kinase activity of hSMG-1.

To support the possibility, the above immunoprecipitation and phosphorylation were repeated, except for the use of another anti-hSMG-1 serum, antiserum L, instead of antiserum C. In this connection, antiserum L was obtained by using as an immunogen an N-terminal fragment [corresponding to the sequence consisting of the 864th to 1059th amino acid residues in the amino acid sequence of hSMG-1 (3657 amino acids)] of hSMG-1, which was expressed in E. coli as a protein (molecular weight=approximately 50 kDa) fused to GST.

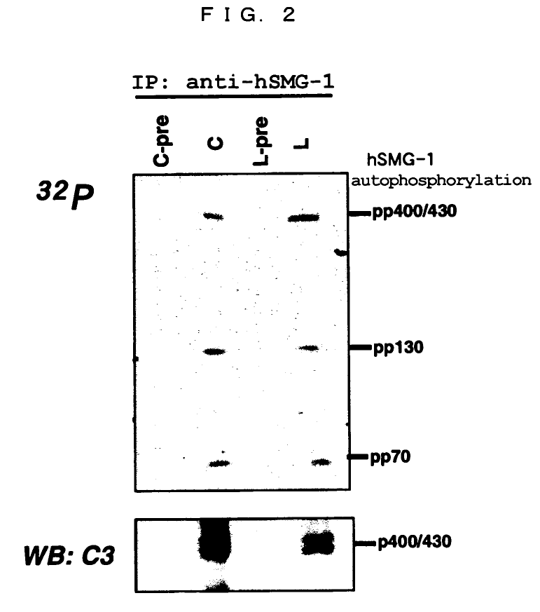

The results are shown in FIG. 2. In FIG. 2, the symbol “³²P” indicates the results obtained by autoradiography, and the symbol “WB:C3” indicates the results obtained by Western blotting using antiserum C. The symbols “C-pre”, “C”, “L-pre”, and “L” indicate the results obtained by immunoprecipitation using the preimmune serum of antiserum C, antiserum C, the preimmune serum of antiserum L, and antiserum L, respectively.

When not only antiserum C, but also antiserum L, obtained by using a different region of hSMG-1 as an immunogen, was used, two proteins of 130 kDa and 70 kDa were detected as phosphorylated proteins. The results increase the above possibility, and exclude the possibility that one or more proteins nonspecifically immunoprecipitated by antiserum C is accidentally phosphorylated by hSMG-1.

### Example 2

### Fractionation of HeLa Cell Extract by Liquid Column Chromatography and Phosphorylation in hSMG-1 Immunocomplex

(1) Preparation of Cell Extract

The following procedures were carried out at 4° C. or less.

HeLa cells were cultured to 3×10⁹ cells in accordance with a conventional method, and collected in PBS by the method described in Example 1(1). To the cells, after removing the supernatant by centrifugation, 35 mL of buffer TG [20 mmol/L Tris-HCl (pH7.5), 2 mmol/L EDTA, 2 mmol/L DTT, 1 mmol/L sodium orthovanadate, and 10% glycerol] supplemented with 0.25% Triton X-100 and protease inhibitors (0.1 mmol/L PMSF, 10 μg/mL aprotinin, and 10 μg/mL leupeptin) was added and stirred gently. The cell suspension was transferred to a Potter type homogenizer, and disrupted by moving a pestle made of polytetrafluoroethylene for 50 strokes in the homogenizer, to extract proteins. The whole was ultracentrifuged at 100000×g for 60 minutes to obtain a cell extract. The cell extract was filtered through a membrane filter (0.22 μm pore size; Millipore) to prepare a sample to be analyzed.

(2) Liquid Column Chromatography

Four anion exchange columns (HiTrapQ; resin volume=5 mL; Pharmacia) were joined, connected to a liquid column chromatography system (FPLC; Pharmacia), and equilibrated with buffer A (Buffer TG supplemented with 0.1% Triton X-100). The sample to be analyzed prepared in Example 2(1) was applied to the column, and the column was thoroughly washed with buffer A. Proteins were eluted from the resins under conditions such that the ratio of buffer B (buffer A supplemented with 1 mol/L sodium chloride) in buffer A was linearly increased from 0% to 60% for 85 minutes (flow rate=1.5 mL/min.). A fraction collector attached to the FPLC system was used to fractionate eluates at a rate of 2 min./fraction (approximately 3 mL) continuously. Further, the degrees of eluted proteins were monitored at an absorbance of 280 nm.

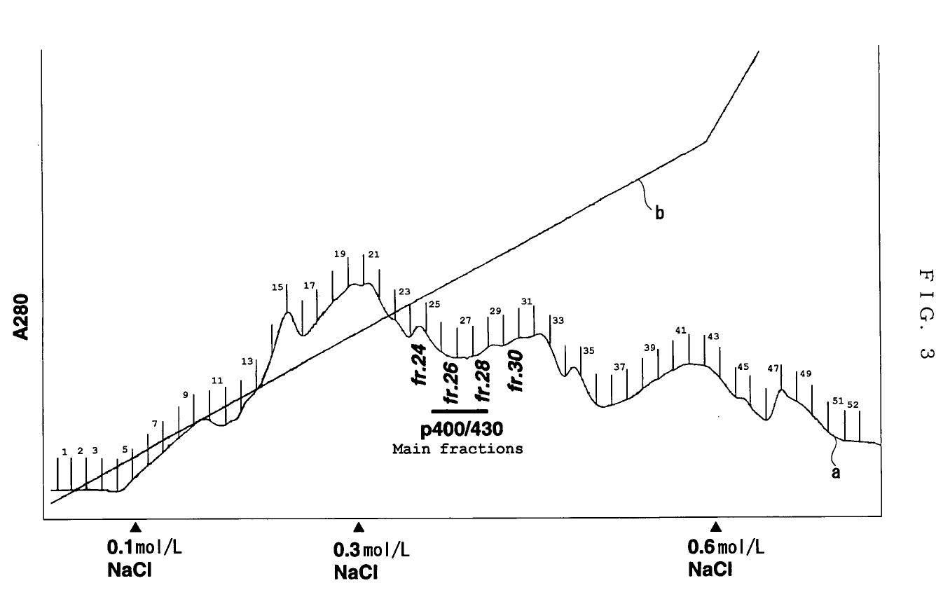

The results are shown in FIG. 3. In FIG. 3, curve a indicates the absorbance at 280 nm (A280), i.e., relative concentration of proteins, and line b indicates a concentration gradient of sodium chloride. The symbol “fr.” means a fraction. Each number following the symbol “fr.”, and each hand-written number described along the curve a is a fraction number.

(3) Immunoprecipitation of hSMG-1 from each Fraction and Phosphorylation

Each fraction (equivalent of 10 μL) was analyzed by Western blotting using anti-hSMG-1 antibody C or anti-hSMG-1 antibody P.

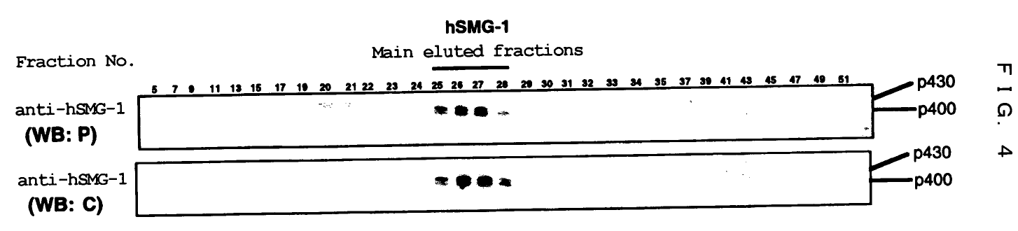

The results are shown in FIG. 4. The symbols “WB:P” and “WB:C” indicate the results obtained by Western blotting using antiserum P and antiserum C, respectively.

It was found that concentrated hSMG-1 was eluted in fractions 25 to 28.

Each fraction (600 μL) was used to carry out immunoprecipitation and phosphorylation by the methods described in Example 1(2) and Example 1(3), except that an amount of the antibody (antiserum C) used in immunoprecipitation was 1 μL/fraction; and that the 2×kinase reaction solution used in phosphorylation did not contain wortmannin, but contained 10 mmol/L MnCl₂ as the divalent metal ion, and further contained 2 μg of a GST-p53(1-58) protein as an exogenous substrate. In this connection, the GST-p53(1-58) protein was a fusion protein of GST and a fragment consisting of the 1st to 58th amino acids in protein p53 (amino acid residues=393). It is known that the 15th Serine residue (Ser15) and the 37th Serine residue (Ser37) in protein p53 locate in a sequence which accords with a phosphorylated consensus motif of hSMG-1, and are phosphorylated by hSMG-1 [Yamashita, Akio, et al, “GENES & DEVELOPMENT”, (USA), 2001, 15, p. 2215-2228].

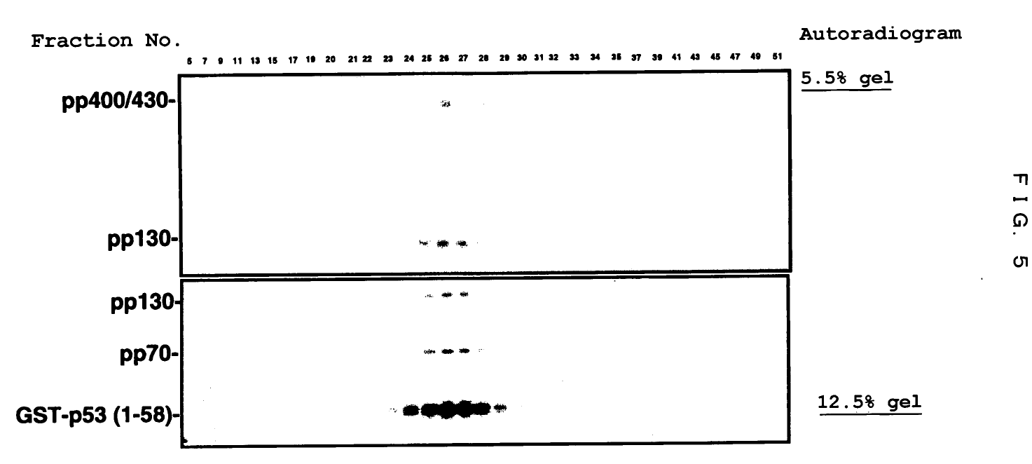

After phosphorylation, the reaction solutions were analyzed by SDS-PAGE and autoradiography. The results are shown in FIG. 5. After the column fractionation was carried out, the autophosphorylation activity and the activity of exogenous-substrate of hSMG-1 were not separated from p130 and p70. The results strongly suggest that hSMG-1 always behaves together with p130 and p70.

Next, fractions 20 to 31 were used to carry out immunoprecipitation and phosphorylation by the methods described in Example 1(2) and Example 1(3), except that the 2×kinase reaction solution used in phosphorylation contained 2 μg of the GST-p53(1-58) protein as an exogenous substrate; the starting solution contained 1 mmol/L ATP, but did not contain radioactive ATP; and the reaction was carried out for 1 hour.

The states of the phosphorylated GST-p53(1-58) protein were analyzed by SDS-PAGE, and Western blotting using an antibody against phosphorylated Ser15 in protein p53 (anti P-Ser15 antibody; Shieh, S. Y. et al., Cell, 91, 325-334, 1997) or an antibody against phosphorylated Ser37 in protein p53 (anti P-Ser37 antibody; Kitagawa, M. et al., EMBO J., 15, 7060-7069, 1996).

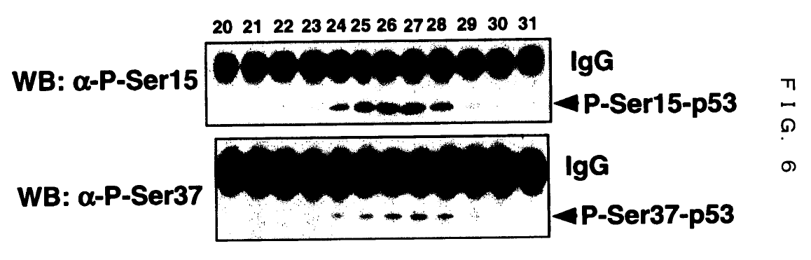

The results are shown in FIG. 6. The symbols “WB:α-P-Ser15” and “WB:α-P-Ser37” indicate the results obtained by Western blotting using the anti P-Ser15 antibody and the anti P-Ser37 antibody, respectively. The symbols “P-Ser15-p53” and “P-Ser37-p53” mean the GST-p53(1-58) proteins phosphorylated at Ser15 and Ser37, respectively.

### Example 3

### Purification of hSMG-1 Complex by Antibody Column and Analysis Thereof

(1) Immunoprecipitation

The HeLa cell extract was prepared by the method described in Example 1(1). To the cell extract, 3 mL (volume) of protein A Sepharose (Amersham-Pharmacia) was added, and stirred at 4° C. for 30 minutes. The whole was centrifuged to obtain the supernatant. To 100 μL of protein A Sepharose, on which 25 μg of anti-hSMG-1 IgG (antibody N) purified from antiserum N by a conventional method, or 25 μg of normal rabbit IgG (Santa Cruz), was previously adsorbed, half of the supernatant was added and stirred at 4° C. for 2 hours. In this connection, antiserum N was obtained by using as an immunogen an N-terminal fragment [corresponding to the sequence consisting of the 1st to 106th amino acid residues in the amino acid sequence of hSMG-1 (3657 amino acids)] of hSMG-1, which was expressed in E. coli as a protein fused to GST.

After the whole was centrifuged to remove the supernatant, the F-lysis solution was newly added and stirred for a short time, and the supernatant was removed by recentrifugation. The step was repeated six times. To the collected protein A Sepharose particles, 100 μL of a 2×SDS-sample buffer was added, and boiled at 100° C. for 4 minutes. The whole was centrifuged to obtain the supernatant as samples to be analyzed.

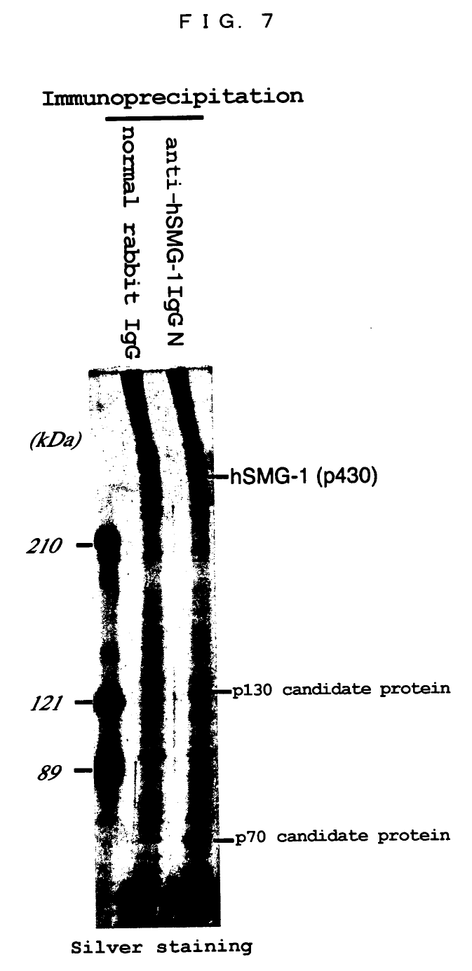

In accordance with conventional methods, 1/20 of the samples were analyzed by SDS-PAGE and silver staining. The results are shown in FIG. 7. In the immunoprecipitate by the anti hSMG-1 IgG antibody, a band was detected at the position of approximately 130 kDa. The band was not detected in the immunoprecipitate by the normal rabbit IgG. In addition, a band, which was considered to be immunoprecipitated hSMG-1, was detected at the position of approximately 430 kDa.

(2) Mass Spectrometry

In accordance with conventional methods, ¼ of the samples to be analyzed were subjected to SDS-PAGE, and transferred to a polyvinylidene difluoride (PVDF) membrane (Immobilon C; Millipore). After the transfer, the membrane was immersed in a Coomassie brilliant blue (CBB) solution for a short time, washed in water, and dried, to observe transferred and stained proteins. A band considered to be 430 kDa and a band considered to be the phosphorylated protein of 130 kDa were detected.

The stained protein of 130 kDa (p130) was excised from the membrane with a knife. In accordance with a known method [for example, Akihiko Iwamatsu, Experimental medicine (Jikken Igaku), Vol. 17, No. 8, 1999, p. ¹³⁵I-1355, “Close-up in experimental methods—Proteome analysis by mass spectrometer-”], the protein was thoroughly digested with protease Lys-C (lysylendopeptidase) on the membrane, and the digested fragments were analyzed by mass spectrometry using MALDI-TOF/MS (matrix assisted laser desorption ionization time-of-flight mass spectrometry).

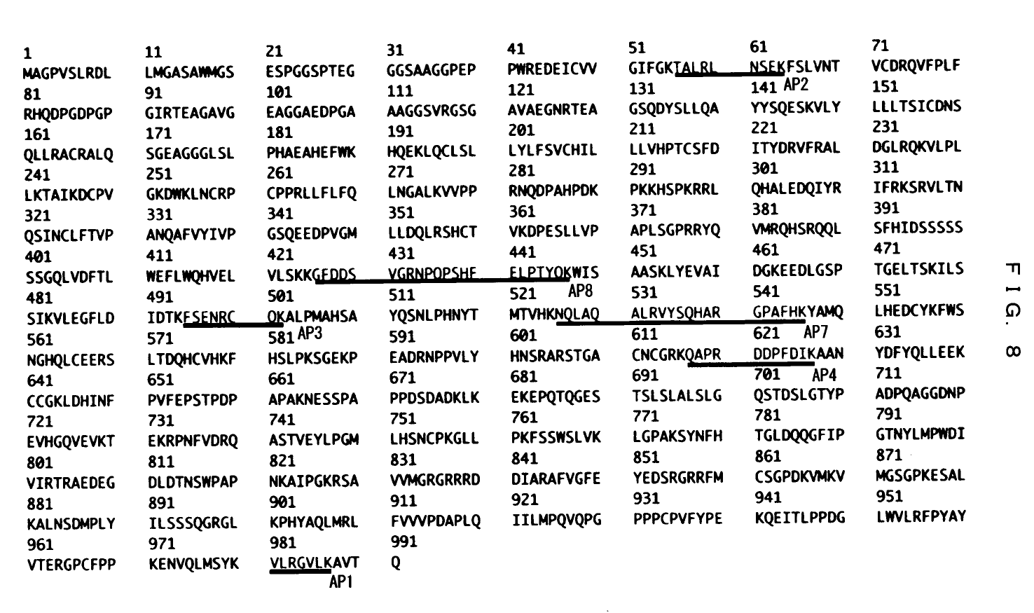

As a result, each mass of eight Lys-C-digested fragments (AP1 to AP8) shown in Table 1 was determined. A search through known databases in the primary structure of proteins was carried out. Among the eight masses, six masses of protein fragments (AP1, AP2, AP3, AP4, AP7, and AP8) accorded with those of Lys-C-digested fragments deduced from a translated product of cDNA clone AK026858.1 registered in known databases. In FIG. 8, the primary structure (the amino acid sequence of SEQ ID NO: 2) of the translated product of AK026858.1, and the positions of sequences corresponding to AP1, AP2, AP3, AP4, AP7, and AP8 are shown. From the results, it was supposed that p130 was the translated product of AK026858.1, and an hSMG-1 binding protein. The translated product of AK026858.1 contains no region homologous to known functional motifs. However, genes which may encode proteins having a high homology with the translated product of AK026858.1 exist in Drosophila (CG6729; homology=41%) and Caenorhabditis elegans (K04B12.3; homology=41%), and thus these proteins are considered to evolutionarily conserved proteins. In this connection, each homology described above is a value determined by BLAST (Basic local alignment search tool; Altschul, S. F. et al., J. Mol. Biol., 215, 403-410, 1990).

TABLE 1

| | Mass | Mass | | |
|---|---|---|---|---|
| Peptide | (measured) | (calculated) | Error | Sequence |
| AP1 | 784.54 | 784.54 | 0.00 | SEQ ID NO: 3 |
| AP2 | 1031.61 | 1031.58 | 0.03 | SEQ ID NO: 4 |
| AP3 | 1069.55 | 1069.47 | 0.08 | SEQ ID NO: 5 |
| AP4 | 1301.71 | 1301.65 | 0.06 | SEQ ID NO: 6 |
| AP5 | 1599.86 | — | — | — |
| AP6 | 2196.10 | — | — | — |
| AP7 | 2392.28 | 2392.27 | 0.01 | SEQ ID NO: 7 |
| AP8 | 2519.26 | 2519.20 | 0.06 | SEQ ID NO: 8 |

### Example 4

### Preparation of Anti-p130 Antibody and Analysis of Expressed Protein

(1) Preparation of Antibody

The AK026858.1 DNA had been identified in the Full-length human cDNA sequencing project (http://www.nedo.go.jp/bio-e/) by Dr. Sugano et al. in the Institute of Medical Science, the University of Tokyo, and the plasmid used in this example, in which the AK026858.1 DNA was inserted into pME18S, was provided from Dr. Sugano. To confirm that the translated product of AK026858.1 is an hSMG-1 binding protein, a specific antibody thereagainst was prepared.

The plasmid containing AK026858.1 was digested with restriction enzymes SmaI and MscI to obtain a DNA fragment of 446 bp (corresponding to the amino acid sequence consisting of the 563rd to 710th amino acids in the amino acid sequence of SEQ ID NO: 2). The DNA fragment was inserted at the SmaI site of E. coli expression vector pGEX-6P-1 (Amersham Pharmacia), and the resulting recombinant plasmid was introduced into E. coli DH5. E. coli strains containing the recombinant plasmid were cultured, and the p130 protein fragment was purified as a protein fused to GST, in accordance with a conventional method. In accordance with a conventional method, rabbits (New Zealand White) were immunized with the purified fusion protein as an immunogen to obtain an antiserum. Further, a specific antibody (anti AK026858.1-translated-product antibody) was separated from the antiserum by a conventional method (affinity purification method) using the immunogen protein.

(2) Expression of p130 in HeLa Cells and Confirmation of hSMG-1 Binding Activity by Immunoprecipitation

The HeLa cell extract was prepared by the method described in Example 1(1), and immunoprecipitation using the normal rabbit serum or the anti-hSMG-1 antiserums (antiserum N, antiserum L, or antiserum C) was carried by the method described in Example 3(1). The cell extract and immunocomplex were subjected to SDS-PAGE, and analyzed by Western blotting using the anti AK026858.1-translated-product antibody prepared in Example 4(1), in accordance with conventional methods.

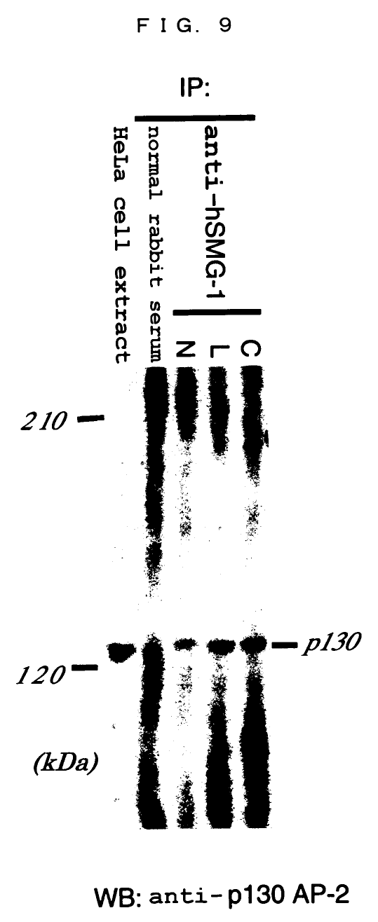

The results are shown in FIG. 9. In FIG. 9, the symbol “IP” means immunoprecipitation. The symbols “N”, “L”, and “C” indicate the results obtained by using antiserum N, antiserum L, and antiserum C, respectively. The symbol “WB” means Western blotting.

First, it was found that the antibodies recognized the translated product of AK026858.1, because the protein having a molecular weight of 130 kDa was specifically detected in the cell extract. Further, the same protein was detected in any case of immunoprecipitation using antiserum N, antiserum L, or antiserum C, but such a protein was not detected in the case of immunoprecipitation using the normal rabbit serum. From the results, it was found that the translated product of AK026858.1 was a binding protein specific to hSMG-1. From the molecular weight thereof, it was strongly suggested that the binding protein was the 130 kDa protein which may be phosphorylated in the hSMG-1 immunoprecipitate.

### Example 5

### Analysis of Phosphorylation of hUPF1 in Cells Wherein Expression of p130 is Suppressed

To confirm that the p130 protein is involved in the intracellular NMD mechanism, the phosphorylation of human UPF1 (hUPF1) was examined in cells wherein an expression of p130 was suppressed by introducing small interfering RNAs (siRNAs) against p130 into cells (HeLa TetOff cells). In this connection, UPF1 is phosporylated by SMG-1 and is an essential factor of NMD, and the phosphorylation of UPF1 is an essential step in the mRNA surveillance [Yamashita, Akio, et al, “GENES & DEVELOPMENT”, (USA), 2001, 15, p. 2215-2228].

Seven (7) siRNAs against p130 used in this example are shown in Tables 2 and 3. Target sequences for designing the p130-specific siRNAs are shown in Table 2, and nucleotide sequences (sense strands) of the p130-specific siRNAs are shown in Table 3. In Table 3, capital letters indicate double-stranded RNA portions (19 nt), and small letters indicate 3′ overhangs (2 nt). The sense and antisense strands of siRNAs Dl to D4 contain UU as the 3′ overhangs, and the sense and antisense strands of siRNAs A1 to A3 contain TT as the 3′ overhangs.

In addition, a control siRNA (QIAGEN), an siRNA against Lamin A/C, and an siRNA against hSMG-1 were used for control or comparison.

TABLE 2

| | | |
|---|---|---|
| | | Target nucleotides |
| siRNA | Target sequence | in SEQ ID NO: 1 |

| | | | |
|---|---|---|---|
| D1 | GCACAAATTTCACTCATTA | (SEQ ID NO: 9) | 1746-1764 |
| D2 | GAAGTTCTCTCTTGTGAAT | (SEQ ID NO: 10) | 204-222 |
| D3 | CAACCAAGCTTTCGTGTAC | (SEQ ID NO: 11) | 1008-1026 |
| D4 | GACTAGAGCTGAAGATGAA | (SEQ ID NO: 12) | 2424-2442 |
| A1 | GGATGAGATCTGCGTTGTG | (SEQ ID NO: 13) | 147-165 |
| A2 | GGTCTTTCCTCTCTTTCGC | (SEQ ID NO: 14) | 240-258 |
| A3 | GGAAAATGTGCAGTTAATG | (SEQ ID NO: 15) | 2928-2946 |

TABLE 3

| | |
|---|---|
| siRNA | Nucleotide sequence |

| | | |
|---|---|---|
| D1 | GCACAAAUUUCACUCAUUAuu | (SEQ ID NO: 16) |
| D2 | GAAGUUCUCUCUUGUGAAUuu | (SEQ ID NO: 17) |
| D3 | CAACCAAGCUUUCGUGUACuu | (SEQ ID NO: 18) |
| D4 | GACUAGAGCUGAAGAUGAAuu | (SEQ ID NO: 19) |
| A1 | GGAUGAGAUCUGCGUUGUGtt | (SEQ ID NO: 20) |
| A2 | GGUCUUUCCUCUCUUUCGCtt | (SEQ ID NO: 21) |
| A3 | GGAAAAUGUGCAGUUAAUGtt | (SEQ ID NO: 22) |

A commercially available reagent (RNAiFect; QIAGEN) was used to introduce the siRNAs into HeLa TetOff cells (Invitrogen) in accordance with a protocol attached thereto. More particularly, HeLa TetOff cells [7.5×10⁴ cells/well (12-well dish)] were transfected with 2 μg of each siRNA, and cultured for 48 hours. The cells were collected and analyzed by Western blotting using an anti-hUPF1 antibody (i.e., antibody specific to nonphosphorylated hUPF1) and an anti-P-hUPF1 antibody (i.e., antibody specific to phosphorylated hUPF1). In addition, antibodies against p130, hSMG-1, hUPF1 phosphatase catalytic subunit PP2Ac, Lamin A/C, or β-actin were used for control or comparison.

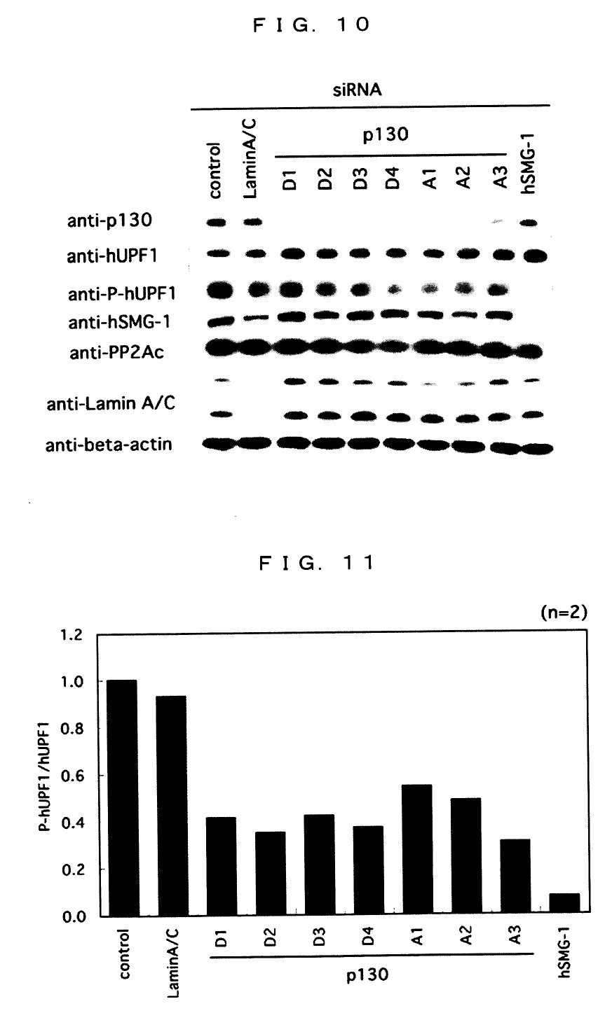

The results are shown in FIG. 10. The procedure was repeated twice. Rates of phosphorylated hUPF1 (P-hUPF1) to hUPF1 (P-hUPF1/hUPF1) are shown, as relative values when the rate in the case of the control siRNA is regarded as 1.0, in FIG. 11.

As shown in FIG. 10, the expression of p130 was remarkably suppressed by transfecting the siRNA against p130 into cells. The phosphorylation of hUPF1 in the cells transfected with the siRNA against p130 was decreased to approximately 40% in comparison with that in the cells transfected with the control siRNA (FIG. 11). In this connection, amounts of hSMG-1 (hUPF1 phosphorylase) and PP2Ac (hUPF1 phosphatase catalytic subunit) were not changed.

As described above, it was confirmed that the phosphorylation of UPF1, which was an essential step in NMD, was decreased by suppressing the expression of p130 (i.e., SMGBP1). That is, it was confirmed that the phosphorylation activity of SMG-1 can be suppressed by suppressing the p130 activity. The results indicate that p130 is involved in, and positively regulates, the intracellular NMD mechanism.

### INDUSTRIAL APPLICABILITY

According to the polypeptide of the present invention, a convenient system for screening an agent for treating and/or preventing a disease caused by one or more PTCs generated by a nonsense mutation can be provided. Further, the polynucleotide, the expression vector, the transformant, and the antibody according to the present invention are useful in producing the polypeptide of the present invention.

Free Text 1N Sequence Listing

Features of “Artificial Sequence” are described in the numeric identifier <223> in the Sequence Listing. More particularly, each of the nucleotide sequences of SEQ ID NOS: 16 to 22 is a sense strand of p130-specific siRNA.

Although the present invention has been described with reference to specific embodiments, various changes and modifications obvious to those skilled in the art are possible without departing from the scope of the appended claims.

1. (1) A polypeptide consisting of the amino acid sequence of SEQ ID NO: 2; (2) a polypeptide exhibiting an SMGBP1 activity and comprising the amino acid sequence of SEQ ID NO: 2; (3) a polypeptide exhibiting an SMGBP1 activity and comprising an amino acid sequence in which one or plural amino acids are deleted, substituted, and/or inserted at one or plural positions in the amino acid sequence of SEQ ID NO: 2; or (4) a polypeptide exhibiting an SMGBP1 activity and comprising an amino acid sequence having a 90% or more homology with that of SEQ ID NO: 2.

2. A polynucleotide encoding the polypeptide according to claim 1.

3. An expression vector comprising the polynucleotide according to claim 2.

4. A transformant comprising the polynucleotide according to claim 2.

5. An antibody or a fragment thereof, which binds to the polypeptide according to claim 1.

6. A knockout non-human animal or a cell thereof, wherein an expression of a gene encoding the polypeptide according to claim 1 is partially or completely suppressed.

7. A method for screening a substance which modifies an SMGBP1 activity of the polypeptide according to claim 1, comprising the steps of:
(1) bringing the polypeptide and SMG-1 into contact with a substance to be tested, under conditions such that the polypeptide exhibits the SMGBP1 activity in the absence of the substance to be tested; and
(2) analyzing whether or not the polypeptide exhibits the SMGBP1 activity.

8. A method for screening a substance which modifies an SMGBP1 activity of the polypeptide according to claim 1, comprising the steps of:
(1) bringing a cell capable of producing the polypeptide and SMG-1 into contact with a substance to be tested, under conditions such that the cell produces the polypeptide in the absence of the substance to be tested; and
(2) analyzing whether or not the polypeptide exhibits the SMGBP1 activity.

9. An agent for suppressing nonsense-mediated mRNA decay, comprising, as an active ingredient, a substance which modifies an SMGBP1 activity of the polypeptide according to claim 1.

10. An agent for treating and/or preventing a disease caused by a premature translation termination codon generated by a nonsense mutation, comprising, as an active ingredient, a substance which modifies an SMGBP1 activity of the polypeptide according to claim 1.

11. An agent for promoting nonsense-mediated mRNA decay, comprising as an active ingredient, a substance which modifies an SMGBP1 activity of the polypeptide according to claim 1.

12. A method for suppressing nonsense-mediated mRNA decay, comprising administering to a subject in need thereof a substance which modifies an SMGBP1 activity of the polypeptide according to claim 1 in an amount effective therefor.

13. A method for treating and/or preventing a disease caused by a premature translation termination codon generated by a nonsense mutation, comprising administering to a subject in need thereof a substance which modifies an SMGBP1 activity of the polypeptide according to claim 1 in an amount effective therefor.

14. A method for promoting nonsense-mediated mRNA decay, comprising administering to a subject in need thereof a substance which modifies an SMGBP1 activity of the polypeptide according to claim 1 in an amount effective therefor.

15. The agent according to claim 9, wherein the substance which modifies an SMGBP1 activity of the polypeptide according to claim 1 is a substance which suppresses an SMGBP1 activity of the polypeptide.

16. The agent according to claim 15, wherein the substance which suppresses an SMGBP1 activity of the polypeptide is (1) an siRNA designed on the basis of an SMGBP1-specific nucleotide sequence consisting of 19 to 21 nucleotides in a nucleotide sequence of SMGBP1, or (2) an expression vector expressing the siRNA in a subject to be administered.

17. The agent according to claim 10, wherein the substance which modifies an SMGBP1 activity of the polypeptide according to claim 1 is a substance which suppresses an SMGBP1 activity of the polypeptide.

18. The agent according to claim 17, wherein the substance which suppresses an SMGBP1 activity of the polypeptide is (1) an siRNA designed on the basis of an SMGBP1-specific nucleotide sequence consisting of 19 to 21 nucleotides in a nucleotide sequence of SMGBP1, or (2) an expression vector expressing the siRNA in a subject to be administered.

19. An siRNA designed on the basis of an SMGBP1-specific nucleotide sequence consisting of 19 to 21 nucleotides in a nucleotide sequence of SMGBP1.

20. The siRNA according to claim 19, wherein the SMGBP1-specific nucleotide sequence is a sequence selected from the group consisting of the sequences of SEQ ID NOS: 9 to 15.

21. The siRNA according to claim 19, wherein a double-stranded RNA portion consists of 1st to 19th nucleotides in a sequence of any one of SEQ ID NOS: 16 to 22.

22. The siRNA according to claim 19, wherein a sense strand consists of a sequence of any one of SEQ ID NOS: 16 to 22.

23. The agent according to claim 11, wherein the substance which modifies an SMGBP1 activity of the polypeptide according to claim 1 is a substance which promotes an SMGBP1 activity of the polypeptide.


SEQUENCE LISTING (22 sequences)

SEQ ID NO: 1 - DNA, 3256 bp, Homo sapiens
  CDS (16)..(2991)
agaacgctctgcactatggctggtcccgtgagcttgcgagaccttctaatgggagcgtcagcctggatgggctctgaaagtcccggagggtcccctactgagggcggagggagcgcggctggcggaccggagcctccatggcgggaggatgagatctgcgttgtgggaatcttcggcaagacggctctacgcctgaattccgagaagttctctcttgtgaatacggtgtgcgaccgacaggtctttcctctctttcgccaccaagatcctggggatccaggacctggaatcagaactgaggctggcgccgtgggtgaggccggtggagccgaggaccctggggctgcagccgggggttcagttcggggaagtggagctgtcgcggaaggtaaccgaactgaggcaggctcccaggactacagccttctgcaggcctactacagtcaggaaagcaaagttctgtatcttctcctcacctccatctgtgacaattcacagcttctccgggcttgtcgggctcttcagagcggggaagctggaggtggactctctttacctcatgcagaagcacacgagttctggaagcatcaagagaagctgcagtgcctcagtctcctttacctattctctgtctgtcatatcttgcttctggtccatcccacttgttcctttgatatcacttatgatcgagtattcagagccctggatgggctgagacagaaggtcctgccgctccttaaaacagccattaaggattgtccagttggcaaagactggaagctaaactgccgaccttgcccacctagactccttttcctctttcaactcaatggagccctcaaggtagtacctcctcggaaccaagacccagctcatccagacaagcccaagaaacattctcccaaaaggaggctgcagcatgccctggaggaccagatctatagaatcttccggaagagtcgtgtcttgactaatcagagcatcaactgcctctttactgtgcctgccaaccaagctttcgtgtacatagtaccgggaagccaggaggaggacccagtaggtatgttgctggaccaacttaggagtcattgtactgtgaaggacccggaatctttgctggtgcctgcacccctttctgggcctaggcgataccaggtgatgaggcagcacagccgacaacaactttcctttcacattgacagcagcagttccagttcttcaggccagctagtggatttcactctttgggaattcctatggcagcatgtggagctagttctaagcaagaaaggtttcgatgacagtgtgggcaggaacccacagccttcccattttgaacttcctacttatcagaagtggatctcagcagcttcaaaactgtatgaggtggctattgatgggaaagaagaggacttggggtcacccactggagagctaacatctaagattttaagcagtattaaagtcttggaaggatttttggatattgacacaaaattctcagaaaaccgatgccaaaaagctttacccatggcccacagtgcctaccagtcaaatttgcctcataattacacaatgactgtccataagaatcagcttgcccaggctcttcgagtgtacagtcaacatgctagaggtccagcatttcacaaatacgccatgcagttacatgaggactgctacaaattttggagcaatggccatcagctctgtgaggagaggagtttaactgatcaacactgtgtgcacaaatttcactcattacctaaatcaggagaaaaaccagaggctgatagaaatccgcctgtgctatatcacaatagccgagctcgatctactggtgcttgcaactgtggaaggaaacaagcacctcgagatgatccctttgatatcaaagcagccaattatgacttctatcagcttctggaagaaaagtgttgtggaaaattggatcatatcaatttcccagtatttgaaccaagtactccagatcctgctcctgctaaaaatgaatcctctcctgctcctccagattcggatgctgataaacttaaagaaaaagaacctcaaacccaaggagagagcacgagcctgagtttagctttgagtttgggccaatccacagatagcttaggtacctatccagctgatccacaagcaggaggagataatccagaagttcatggtcaagtagaagtgaaaactgagaagagaccaaacttcgttgatcgacaggcatccacagttgagtatctcccaggcatgctacattcaaattgccctaaaggtctcctacccaaattctccagctggtctttggttaaactaggccctgctaagtcttataactttcatacaggtttggaccaacagggctttattccaggaacaaattatcttatgccttgggacattgtcatcaggactagagctgaagatgaaggagacttagacacaaactcttggcctgctccaaataaagctattcctggaaagagaagtgcggttgtaatgggaagaggaagacggcgagatgacatagctcgagcttttgtgggctttgaatatgaagactctcgaggtcggagattcatgtgctctgggcctgacaaagtaatgaaagtaatgggaagtgggccaaaggaatcagctttaaaagccctaaatagtgacatgcccttatatattctgtcatcctctcaaggtagagggctgaaacctcattatgctcaacttatgaggctttttgttgtggttcctgatgctcctttgcagataatactaatgcctcaggttcagccaggcccaccaccatgtccggtattctacccagaaaaacaagaaatcacccttccacctgatggcctttgggttttgagatttccttatgcatatgtgactgagagaggaccttgtttcccccctaaggaaaatgtgcagttaatgagctacaaggtgctccgtggggttcttaaggcagtaacacaataagtgttttccagccagttcaatcctatacttgagctggtttttgtttttggtatttgtggctgtgtttttggtatgtgtttactgtgttttcccaaatccaaaatgtgttttggttacaggagttcagtatttggaaactaatttgttctactttttcattatattgttgatatttattgtaaacgtgactacaactttctaataaagattggttgttctagaaaagaatataaaatacatggatgataaaaaaaaaaaaaaaaaa

SEQ ID NO: 2 - protein, 991 aa, Homo sapiens
MAGPVSLRDLLMGASAWMGSESPGGSPTEGGGSAAGGPEPPWREDEICVVGIFGKTALRLNSEKFSLVNTVCDRQVFPLFRHQDPGDPGPGIRTEAGAVGEAGGAEDPGAAAGGSVRGSGAVAEGNRTEAGSQDYSLLQAYYSQESKVLYLLLTSICDNSQLLRACRALQSGEAGGGLSLPHAEAHEFWKHQEKLQCLSLLYLFSVCHILLLVHPTCSFDITYDRVFRALDGLRQKVLPLLKTAIKDCPVGKDWKLNCRPCPPRLLFLFQLNGALKVVPPRNQDPAHPDKPKKHSPKRRLQHALEDQIYRIFRKSRVLTNQSINCLFTVPANQAFVYIVPGSQEEDPVGMLLDQLRSHCTVKDPESLLVPAPLSGPRRYQVMRQHSRQQLSFHIDSSSSSSSGQLVDFTLWEFLWQHVELVLSKKGFDDSVGRNPQPSHFELPTYQKWISAASKLYEVAIDGKEEDLGSPTGELTSKILSSIKVLEGFLDIDTKFSENRCQKALPMAHSAYQSNLPHNYTMTVHKNQLAQALRVYSQHARGPAFHKYAMQLHEDCYKFWSNGHQLCEERSLTDQHCVHKFHSLPKSGEKPEADRNPPVLYHNSRARSTGACNCGRKQAPRDDPFDIKAANYDFYQLLEEKCCGKLDHINFPVFEPSTPDPAPAKNESSPAPPDSDADKLKEKEPQTQGESTSLSLALSLGQSTDSLGTYPADPQAGGDNPEVHGQVEVKTEKRPNFVDRQASTVEYLPGMLHSNCPKGLLPKFSSWSLVKLGPAKSYNFHTGLDQQGFIPGTNYLMPWDIVIRTRAEDEGDLDTNSWPAPNKAIPGKRSAVVMGRGRRRDDIARAFVGFEYEDSRGRRFMCSGPDKVMKVMGSGPKESALKALNSDMPLYILSSSQGRGLKPHYAQLMRLFVVVPDAPLQIILMPQVQPGPPPCPVFYPEKQEITLPPDGLWVLRFPYAYVTERGPCFPPKENVQLMSYKVLRGVLKAVTQ

SEQ ID NO: 3 - protein, 7 aa, Homo sapiens
VLRGVLK

SEQ ID NO: 4 - protein, 9 aa, Homo sapiens
TALRLNSEK

SEQ ID NO: 5 - protein, 8 aa, Homo sapiens
FSENRCQK

SEQ ID NO: 6 - protein, 11 aa, Homo sapiens
QAPRDDPFDIK

SEQ ID NO: 7 - protein, 21 aa, Homo sapiens
NQLAQALRVYSQHARGPAFHK

SEQ ID NO: 8 - protein, 22 aa, Homo sapiens
GFDDSVGRNPQPSHFELPTYQK

SEQ ID NO: 9 - DNA, 19 bp, Homo sapiens
gcacaaatttcactcatta

SEQ ID NO: 10 - DNA, 19 bp, Homo sapiens
gaagttctctcttgtgaat

SEQ ID NO: 11 - DNA, 19 bp, Homo sapiens
caaccaagctttcgtgtac

SEQ ID NO: 12 - DNA, 19 bp, Homo sapiens
gactagagctgaagatgaa

SEQ ID NO: 13 - DNA, 19 bp, Homo sapiens
ggatgagatctgcgttgtg

SEQ ID NO: 14 - DNA, 19 bp, Homo sapiens
ggtctttcctctctttcgc

SEQ ID NO: 15 - DNA, 19 bp, Homo sapiens
ggaaaatgtgcagttaatg

SEQ ID NO: 16 - RNA, 21 nt, Artificial Sequence
  Description of Artificial Sequence sense strand of p130-specific siRNA
gcacaaauuucacucauuauu

SEQ ID NO: 17 - RNA, 21 nt, Artificial Sequence
  Description of Artificial Sequence sense strand of p130-specific siRNA
gaaguucucucuugugaauuu

SEQ ID NO: 18 - RNA, 21 nt, Artificial Sequence
  Description of Artificial Sequence sense strand of p130-specific siRNA
caaccaagcuuucguguacuu

SEQ ID NO: 19 - RNA, 21 nt, Artificial Sequence
  Description of Artificial Sequence sense strand of p130-specific siRNA
gacuagagcugaagaugaauu

SEQ ID NO: 20 - DNA, 21 bp, Artificial Sequence
  Description of Artificial Sequence sense strand of p130-specific siRNA
ggaugagaucugcguugugtt

SEQ ID NO: 21 - DNA, 21 bp, Artificial Sequence
  Description of Artificial Sequence sense strand of p130-specific siRNA
ggucuuuccucucuuucgctt

SEQ ID NO: 22 - DNA, 21 bp, Artificial Sequence
  Description of Artificial Sequence sense strand of p130-specific siRNA
ggaaaaugugcaguuaaugtt